(19) United States
(12) Patent Application Publication
(10) Pub. No.: US 2023/0189833 A1
(43) Pub. Date: Jun. 22, 2023

(54) DAIRY SUBSTITUTES PRODUCED IN PLANT-BASED SYSTEMS AND METHOD THEREOF

(71) Applicant: Alejandro BARBARINI, Santa Fe (AR)

(72) Inventor: Alejandro BARBARINI, Santa Fe (AR)

(21) Appl. No.: 17/913,690

(22) PCT Filed: May 23, 2021
(86) PCT No.: PCT/IL2021/050601

§ 371 Date: Sep. 22, 2022

Related U.S. Application Data
(60) Provisional application No. 62/993,092, filed on Mar. 23, 2020.

(51) Int. Cl. A23C 11/06; A23C 20/02; A23J 1/00; A23J 3/14; A23L 7/104

(52) U.S. Cl. CPC A23C 11/06; A23C 20/02; A23J 1/006; A23J 3/14; A23L 7/104

(57) ABSTRACT

The present invention provides plant-based dairy substitutes and methods thereof. Those substitutes may be a cheese substitute, a yogurt substitute, a coffee creamer substitute and more. The present invention discloses a plant cell culture, preferably, carrot cells, which expresses transgenic casein proteins. This unique casein-expressing culture is then transformed into a slurry, which serves as the platform for the production of the plant-based dairy substitutes. Furthermore, those dairy substitutes are highly nutritious, as they contain beneficial ingredients derived from the plant cells (such as beta-carotene) in addition to high protein (casein) content. The application of carrot cell slurry containing casein proteins to those dairy substitutes provides organoleptic and physicochemical properties enhanced or similar to conventional dairy products. The carrot cells expressing casein proteins can be conserved in a powder form, as the plant cells successfully encapsulate the casein proteins, thus protecting them from physicochemical conditions, such as spray drying.

### CROSS REFERENCE TO RELATED APPLICATIONS

This application is a National Phase of PCT Patent Application No. PCT/IL2021/050601 having International filing date of May 23, 2021, which claims the benefit of priority of U.S. Provisional Patent Application No. 62/993,092, filed Mar. 23, 2020, the contents of which are all incorporated herein by reference in their entirety.

### SEQUENCE LISTING

The contents of the electronic sequence listing (BARBARINI_2400A02PCT_US-SEQ_Listing_PatentIn); Size: 3,963 bytes; and Date of Creation: Mar. 9, 2023) is herein incorporated by reference in its entirety.

### FIELD OF THE INVENTION

The present disclosure relates to plant-based dairy substitutes. More particularly, these dairy substitutes are foodstuffs, such as cheese and yogurt alternatives, comprising casein proteins expressed in plant cell cultures and characterized by enhanced nutritional values and similar organoleptic properties compared to dairy products.

### BACKGROUND OF THE INVENTION

In light of the growing population, which is estimated to reach approximately 10 billion people in the upcoming decades, and given the significant climatic changes affecting yield and quality of agricultural crops, researchers attempt to create new techniques to produce more nutritious and sustainable food products, which would also be environmentally friendly and cruelty free. Such foodstuffs may base completely on plant materials (for instance, meat and milk alternatives made of plant proteins or fibers) or on animal proteins or tissues generated in laboratories (meat products referred to as ‘clean meat’ or ‘cultured meat’). Those notions have been gaining a lot of attention in recent years, as topics such as veganism and animal welfare have become increasingly popular. In fact, it is believed that in the upcoming decade, more people will increase their consumption of plant-based products, mainly cheese and meat alternatives.

However, these products are still considered rather costly, and their taste and texture still does not fully resemble the conventional animal- and dairy-based products.

Expression and production of commercially mass-produced proteins for the pharmaceutical and food industries have also improved in recent decades in terms of yield and functionality. The production of heterologous proteins in bio-producers using genetic engineering and emerging biotechnological techniques is now widely used, mainly for scientific and medical purposes. Those proteins are known as ‘recombinant’ or ‘transgenic’ proteins. Currently and due to the development of biological techniques allowing to overcome interspecies barriers, a wide range of protein expression systems is utilized, including bacterial, fungal, algal, insectile, plant and mammalian cells (see “Production of Recombinant Proteins in Plant Cells”, S. V. Gerasimovaa et al., Russian Journal of Plant Physiology, 2016).

Plant-based systems are considered a valuable platform for the production of recombinant proteins, as a result of their well-documented potential for the flexible, low-cost production of high-quality, bioactive products. Plant-based platforms are arising as an important alternative to traditional fermenter-based systems for safe and cost-effective recombinant protein production. Although downstream processing costs are comparable to those of microbial and mammalian cells, the lower up-front investment required for commercial production in plants and the potential economy of scale, provided by cultivation over large areas, are key advantages (see “A Comparative Analysis of Recombinant Protein Expression in Different Biofactories: Bacteria, Insect Cells and Plant Systems”, Elisa Gecchele et al., Journal of Visualized Experiments, 97, p. 1-8, 2015).

In addition, plant-based systems have numerous other advantages as follows: (i) they are distinguished by diversity and plasticity (varying from hairy roots and cell suspension cultures of a fixed volume and high purity to transgenic plants cultivated in large areas); (ii) they are free of dangerous pathogens and toxins found in bacterial- and mammalian-based systems; (iii) they can be cultivated under aseptic conditions using classical fermentation technology; (iv) they are easy to scale-up for manufacturing; (v) they sustain complex post-translational modifications (such as glycosylation) characteristic to eukaryotic proteins; and (vi) the regulatory requirements are similar to those established for well-characterized production systems based on microbial and mammalian cells (see “Putting the spotlight back on plant suspension cultures”, Santos Rita B. et al., Front Plant Sci.; 7:297, Mar. 11, 2016).

More specifically, plant cell suspension cultures have several additional benefits, rendering them even more advantageous in comparison to whole transgenic plants. Suspension cultures are completely devoid of risks such as unpredicted weather, pests, soil infections and gene flow from other plants in the environment. Moreover, due to the short growth cycles of suspension cultured cells, the timescale needed to produce recombinant proteins in plant cell culture can be counted in days compared with months needed for the production in transgenic plants. In addition, growing plant cells in sterile and controlled environments, such as the bioreactor system, allows for precise control over cell growth conditions, batch-to-batch product consistency, utilization of chemically inducible systems and more.

Similar to microbial fermentation, plant cells have relatively rapid doubling times (as fast as 16 hours) and can grow in simple synthetic media using conventional bioreactors.

EP patent 2617294A1 discloses food and food additive compositions comprising one or more human milk proteins produced in the seeds of a transgenic plant and methods of making the same. The invention is further directed to improved infant formula comprising such food supplement composition. The seed-produced human milk protein is selected from the group consisting of human lactoferrin (LF), lysozyme (LZ), lactoperoxidase (LP), immunoglobulins, EGF, IGF-I, lactoadherin, kappa-casein, haptocorrin, alpha-1-antitrypsin, albumin, alpha-lactalbumin, beta-lactoglobulin, alpha-, beta- and kappa-caseins, serum albumin and lipase.

U.S. Pat. No. 5,942,274A discloses a human infant formula sufficient to meet the nutritional requirements of a human infant, comprising proteins having substantially the same amino acid sequence and biological properties as human alpha-lactalbumin and human beta-casein. The proteins may be produced from microorganisms, particularly E. coli. A recombinant DNA segment comprising a human milk protein encoding gene; a promoter sequence directing the transcription of the gene, where the promoter sequence is different from the promoter sequence for the gene in the human organism; and a terminator site for the human milk protein encoding gene. A microorganism containing a recombinant DNA segment comprising a human milk protein encoding gene; a promoter sequence directing the transcription of the gene; and a terminator site for the gene.

US patent 20170273328 discloses dairy substitutes, methods of manufacturing the same, and compositions comprising animal-free milk fats and proteins for food applications, such as milk, butter, cheese, yogurt, and cream The disclosed compositions comprise one or more recombinant proteins selected from the group consisting of a β-lactoglobulin protein, a κ-casein protein, an α-lactalbumin protein, a β-casein protein, an α-S2-casein protein, an α-S1-casein protein, and a serum albumin protein, wherein at least one of the one or more recombinant proteins comprises a sequence that is at least 70% identical to the bovine protein amino acid sequence, and is produced in a fungal cell.

R. PRIBYLOVA et al. disclose transgenic potato plants producing a human lactic β-casein which might also be significant for nourishment. In spite of the relatively low amount of casein produced by potato plants, the disclosed experiment indicates that casein can be expressed in edible crops. Human β-casein produced by plants might be used in the future for the production of human milk proteins such as lactoferrin and lysozyme or for preparation of baby food with increased nutritional value and preventive effects against gastric and intestinal dysfunctions in children (see “Genetically modified potato plants in nutrition and prevention of diseases in humans and animals: a review.” R. PRIBYLOVA, I. PAVLIK, M. BARTOS, Veterinarni Medicina, 51, 2006 (5): 212-223).

In view of the prior art and given the various challenges described above, there is still an unmet long-felt need for plant-based dairy products and method thereof, characterized by enhanced nutritional values and organoleptic properties similar to conventional dairy products.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention.

FIG. 1 schematically depicting the method for producing casein-expressing plant cell suspension powder and slurry for manufacturing the dairy substitutes of the present invention;

FIG. 2 schematically depicting the method for producing the plant-based cheese substitute as disclosed in the present invention; and

FIG. 3 schematically depicting the method for producing the plant-based yogurt substitute as disclosed in the present invention.

### SUMMARY OF THE INVENTION

It is one object of the present invention to disclose a plant-based dairy substitute comprising:
a. a slurry of transgenic plant cells expressing at least one form of casein;
b. water;
c. at least one chemical;
d. at least one food additive;
e. at least one vegetable oil;
f. at least one saccharide;
g. at least one vegetable protein; and
h. at least one strain of lactic bacteria;

wherein said slurry is configured to be fermented by said lactic bacteria, thereby producing a plant-based dairy substitute exhibiting organoleptic and physicochemical properties characteristic of dairy products of animal origin.

It is another object of the present invention to disclose the plant-based dairy substitute of the above, wherein said substitute is selected from a group consisting of cheese, yogurt, cream, custard, ice cream, coffee creamers and emulsifiers for the food industry.

It is another object of the present invention to disclose the plant-based dairy substitute of the above, wherein said at least one form of casein is selected from a group consisting of bovine alpha S1 casein, bovine kappa casein, bovine alpha S2 casein, bovine beta-casein, and any combination thereof.

It is another object of the present invention to disclose the plant-based dairy substitute of the above, wherein said transgenic plant cells are selected from a group consisting of cell suspension cultures, hairy root cultures, transgenic plants and any combination thereof.

It is another object of the present invention to disclose the plant-based dairy substitute of the above, wherein said transgenic plant cells are selected from a group consisting of carrot cells, rice cells, beetroot cells, tobacco cells, potato cells, sweet potato cells, tomato cells, Arabidopsis cells, Nicotiana benthamiana cells, cassava cells, kohlrabi cells, parsley cells, horseradish cells, jackfruit cells, Anchusa officinalis cells and any combination thereof.

It is another object of the present invention to disclose the plant-based dairy substitute of the above, wherein said at least one chemical is selected from the group consisting of calcium salts, potassium phosphate dibasic, monobasic potassium phosphate and any combination thereof.

It is another object of the present invention to disclose the plant-based dairy substitute of the above, wherein said at least one food additive is selected from the group consisting of stabilizers, emulsifiers, anticaking agents, salts, yeast extract, flavorings, antifoaming agents, antioxidants, bulking agents, colorants, humectants, preservatives, sweeteners, vitamins, hydrocolloids, thickeners and any combination thereof.

It is another object of the present invention to disclose the plant-based dairy substitute of the above, wherein said at least one vegetable oil is selected from a group consisting of coconut oil, canola oil, corn oil, olive oil, cottonseed oil, palm oil, peanut oil, sesame oil, soybean oil, grapeseed oil sunflower oil and any combination thereof.

It is another object of the present invention to disclose the plant-based dairy substitute of the above, wherein said vegetable oil comprises about 4%-15% of said plant-based dairy substitute.

It is another object of the present invention to disclose the plant-based dairy substitute of the above, wherein said at least one vegetable protein is selected from a group consisting of nuts, grains, seeds, fruits, tubers and any combination thereof.

It is another object of the present invention to disclose the plant-based dairy substitute of the above, wherein said at least one vegetable protein is selected from a group consisting of cashew, almonds, peanuts, walnuts, brazil nuts, rice, wheat, oat, rye, corn, quinoa, lentil, sesame, chia, pea, chickpea soybean, fava bean, mung bean, pumpkin seeds, sunflower seeds, flaxseeds, potato, cassava, yam and any combination thereof.

It is another object of the present invention to disclose the plant-based dairy substitute of the above, wherein said at least one vegetable protein comprises about 1%-10% of said plant-based cheese.

It is another object of the present invention to disclose the plant-based dairy substitute of the above, wherein said at least one saccharide is selected from a group consisting of starch, modified starch, non-modified starch, corn starch, potato starch, rice starch, tapioca starch, maltodextrin, inulin, glucose, sucrose, fructose, dextrose, inverted sugar and any combination thereof.

It is another object of the present invention to disclose the plant-based dairy substitute of the above, wherein said at least one strain of lactic bacteria is selected from a group consisting of Aerococcus, Enterococcus, Lactobacillus, Lactococcus, Leuconostoc, Oenococcus, Pediococcus, Streptococcus and any combination thereof.

It is another object of the present invention to disclose the plant-based dairy substitute of the above, wherein said lactic bacteria are selected from the group consisting of Streptococcus thermophilus, Lactobacillus helveticus, Lactobacillus bulgaricus, Lactococcus lactis, Lactococcus cremoris, Lactococcus diacetylactis, Lactobacillus acidophilus, Lactobacillus casei, Lactobacillus lactis, Lactobacillus helveticus, Lactobacillus bulgaricus Bifidobacterium and any combination thereof.

It is another object of the present invention to disclose the plant-based dairy substitute of the above, wherein said organoleptic properties are selected from a group consisting of texture, consistency, appearance, taste, odor, flavor, aroma, touch, mouthfeel and any combination thereof.

It is another object of the present invention to disclose the plant-based dairy substitute of the above, wherein said physicochemical properties are selected from a group consisting of strength, firmness, tightness, resilience, rheological parameters, moisture content, viscosity, adhesiveness, cohesiveness, degradation rate, solvation, porosity, electrical charge and any combination thereof.

It is another object of the present invention to disclose the plant-based dairy substitute of the above, wherein said plant-based dairy substitute comprises at least 5 milligrams beta-carotene per 1 Liter.

It is another object of the present invention to disclose the plant-based dairy substitute of the above, wherein said transgenic plant cells are configured to be spray-dried into a plant cell powder, prior to forming said slurry.

It is another object of the present invention to disclose the plant-based dairy substitute of the above, wherein said plant cell powder is storable without refrigeration at about 25° C. for about 6 months.

It is another object of the present invention to disclose a method for producing a plant-based cheese substitute comprising steps of:
a. obtaining a slurry of transgenic plant cells expressing at least one form of casein;
b. dissolving said slurry of transgenic plant cells;
c. forming casein micelle solution;
d. stirring said casein micelle solution;
e. incubating said casein micelle solution with at least one rennet-forming enzymes to form a rennet;
f. filtering said rennet;
g. resuspending said rennet in water to form a solution;
h. adding at least one food ingredient to said solution;
i. mixing said solution;
j. adding at least one vegetable oil to said solution;
k. incubating said solution with at least one strain of pre-activated lactic bacteria to form a fermented preparation;
l. sieving said fermented preparation;
m. adding at least one food additive to said fermented preparation;
n. homogenizing said fermented preparation; and
o. packing said fermented preparation,

wherein, said plant-based cheese substitute is characterized in organoleptic properties and physicochemical properties characteristic of conventional cheese products of animal origin.

It is another object of the present invention to disclose the method for producing a plant-based cheese substitute of the above, wherein said at least one form of casein is selected from a group consisting of bovine alpha S1 casein, bovine kappa casein, bovine alpha S2 casein, bovine beta-casein, and any combination thereof.

It is another object of the present invention to disclose the method for producing a plant-based cheese substitute of the above, wherein said plant cells are selected from a group consisting of carrot cells, rice cells, beetroot cells, tobacco cells, potato cells, sweet potato cells, tomato cells, Arabidopsis cells, Nicotiana benthamiana cells, cassava cells, kohlrabi cells, parsley cells, horseradish cells, jackfruit cells, Anchusa officinalis cells and any combination thereof.

It is another object of the present invention to disclose the method for producing a plant-based cheese substitute of the above, wherein said forming casein micelle solution is obtained by the addition of about 10% 0.2 M CaCl₂ and about 5% K₂HPO₄.

It is another object of the present invention to disclose the method for producing a plant-based cheese substitute of the above, wherein said at least one rennet-forming enzymes is chymosin.

It is another object of the present invention to disclose the method for producing a plant-based cheese substitute of the above, wherein said at least one food ingredient is selected form a group consisting of plant fibers, starch, saccharides, yeast extracts, amino acids, proteins from a plant origin and any combination thereof.

It is another object of the present invention to disclose the method for producing a plant-based cheese substitute of the above, wherein said at least one vegetable oil is selected from a group consisting of coconut oil, canola oil, corn oil, olive oil, cottonseed oil, palm oil, peanut oil, sesame oil, soybean oil, grapeseed oil sunflower oil and any combination thereof.

It is another object of the present invention to disclose the method for producing a plant-based cheese substitute of the above, wherein said at least one strain of pre-activated lactic bacteria are selected from the group consisting of Streptococcus thermophilus, Lactobacillus helveticus, Lactobacillus bulgaricus, Lactococcus lactis, Lactococcus cremoris, Lactococcus diacetylactis, Lactobacillus acidophilus, Lactobacillus casei, Lactobacillus lactis, Lactobacillus helveticus, Lactobacillus bulgaricus Bifidobacterium and any combination thereof.

It is another object of the present invention to disclose the method for producing a plant-based cheese substitute of the above, wherein said organoleptic properties are selected from a group consisting of texture, consistency, appearance, taste, odor, flavor, aroma, touch, mouthfeel and any combination thereof.

It is another object of the present invention to disclose the method for producing a plant-based cheese substitute of the above, wherein said physicochemical properties are selected from a group consisting of strength, firmness, tightness, resilience, rheological parameters, moisture content, viscosity, adhesiveness, cohesiveness, degradation rate, solvation, porosity, electrical charge and any combination thereof.

It is another object of the present invention to disclose a method for producing a plant-based yogurt substitute comprising steps of:
a. obtaining a slurry of transgenic plant cells expressing at least one form of casein;
b. adding said slurry to a supernatant of pre-hydrated, pre-filtered cereal suspension;
c. forming casein micelle solution;
d. stirring said casein micelle solution;
e. adding at least one vegetable oil and at least one saccharide;
f. homogenizing said solution;
g. heating said solution to about 65° C.;
h. cooling down said solution to about 42° C.;
i. incubating said solution with at least one strain of pre-activated lactic bacteria to form a fermented preparation;
j. adding at least one food additive to said fermented preparation;
k. mixing said fermented preparation; and
l. packing said fermented preparation.

wherein, said plant-based yogurt substitute is characterized in organoleptic properties and physicochemical properties characteristic of conventional yogurt products of animal origin.

It is another object of the present invention to disclose the method for producing a plant-based yogurt substitute of the above, wherein said at least one form of casein is selected from a group consisting of bovine alpha S1 casein, bovine kappa casein, bovine alpha S2 casein, bovine beta-casein, and any combination thereof.

It is another object of the present invention to disclose the method for producing a plant-based yogurt substitute of the above, wherein said plant cells are selected from a group consisting of carrot cells, rice cells, beetroot cells, tobacco cells, potato cells, sweet potato cells, tomato cells, Arabidopsis cells, Nicotiana benthamiana cells, cassava cells, kohlrabi cells, parsley cells, horseradish cells, jackfruit cells, Anchusa officinalis cells and any combination thereof.

It is another object of the present invention to disclose the method for producing a plant-based yogurt substitute of the above, wherein said cereal is selected from a group consisting of oat, wheat rye, spelt, triticale and any combination thereof.

It is another object of the present invention to disclose the method for producing a plant-based yogurt substitute of the above, wherein said forming casein micelle solution is obtained by the addition of about 10% 0.2 M CaCl₂ and about 5% K₂HPO₄.

It is another object of the present invention to disclose the method for producing a plant-based yogurt substitute of the above, wherein said at least one vegetable oil is selected from a group consisting of coconut oil, canola oil, corn oil, olive oil, cottonseed oil, palm oil, peanut oil, sesame oil, soybean oil, grapeseed oil sunflower oil and any combination thereof.

It is another object of the present invention to disclose the method for producing a plant-based yogurt substitute of the above, wherein said at least one saccharide is selected from a group consisting of starch, modified starch, non-modified starch, corn starch, potato starch, rice starch, tapioca starch, maltodextrin, inulin, glucose, sucrose, fructose, dextrose, inverted sugar and any combination thereof.

It is another object of the present invention to disclose the method for producing a plant-based yogurt substitute of the above, wherein said at least one strain of pre-activated lactic bacteria are selected from the group consisting of Streptococcus thermophilus, Lactobacillus helveticus, Lactobacillus bulgaricus, Lactococcus lactis, Lactococcus cremoris, Lactococcus diacetylactis, Lactobacillus acidophilus, Lactobacillus casei, Lactobacillus lactis, Lactobacillus helveticus, Lactobacillus bulgaricus Bifidobacterium and any combination thereof.

It is another object of the present invention to disclose the method for producing a plant-based yogurt substitute of the above, wherein said at least one food additive is a flavoring.

It is another object of the present invention to disclose the method for producing a plant-based yogurt substitute of the above, wherein said organoleptic properties are selected from a group consisting of texture, consistency, appearance, taste, odor, flavor, aroma, touch, mouthfeel and any combination thereof.

It is another object of the present invention to disclose the method for producing a plant-based yogurt substitute of the above, wherein said physicochemical properties are selected from a group consisting of strength, firmness, tightness, resilience, rheological parameters, moisture content, viscosity, adhesiveness, cohesiveness, degradation rate, solvation, porosity, electrical charge and any combination thereof.

It is another object of the present invention to disclose a slurry comprising plant cells expressing at least one form of casein for use in the production of foodstuffs, food ingredients and beverages.

### DETAILED DESCRIPTION OF THE PREFERRED EMBODIMENTS

The following description is provided, alongside all chapters of the present invention, so as to enable any person skilled in the art to make use of the invention and sets forth the best modes contemplated by the inventor of carrying out this invention. Various modifications, however, are adapted to remain apparent to those skilled in the art, since the generic principles of the present invention have been defined specifically to provide plant-based cheese products made of transgenic casein proteins, expressed in plant suspension cultures and methods thereof.

As used herein, the term “about” refers to any value being up to 25% lower or greater the defined measure.

As used herein, the term “plant-based dairy substitute” refers to any consumable product, beverage or foodstuff, which is supposed to mimic the appearance, taste, odor, texture, mouthfeel and physicochemical properties of similar products of animal origins (dairy products). Plant-based dairy substitutes are made of either plant proteins or from mammalian proteins which are produced and expressed in non-animal systems under controlled conditions in laboratories, eliminating the need to slaughter or mistreat animals. In the context of the present invention, animal protein (caseins) are expressed in plant cell cultures. The plant cells are modified to produce the end products, which may be cheese, yogurt, custard, ice cream, coffee creamers or cooking cream. In other words, the end products (the dairy substitutes) comprise both casein and plant materials and ingredients.

As used herein, the term “dairy substitute/alternative/analogue” refers to any consumable product or foodstuff, which is not made from animal, parts or derivatives thereof, and is meant to replace animal-based products in one's diet by attempting to mimic or equal the nutritional values, or organoleptic/physicochemical properties of the animal-based products.

As used herein, the term “conventional dairy product/conventional cheese/conventional yogurt/conventional coffee creamer” refers to a dairy product (cheese/yogurt/coffee creamer) which is produced from an animal source, and thus, are not meant to be consumed by vegan or vegetarian populations.

As used herein, the term “plant-based cheese substitute” refers to any consumable product produced by the methods disclosed in the present application using a plant cell slurry expressing transgenic casein proteins, and appearing, tasting, smelling and physicochemically behaving like a conventional cheese product.

Conventional cheese is a dairy product, derived from animal milk and produced in a wide range of flavors, textures and forms by coagulation of the milk protein casein. Cheese comprises proteins and fat from milk, usually the milk of cows, buffalo, goats, or sheep. During production, the milk is usually acidified and the enzymes of rennet (or bacterial enzymes with similar activity) are added to cause the milk proteins (casein) to coagulate. The solids (curd) are separated from the liquid (whey) and pressed into the final form.

Depending on the type of milk used, and fermentation and after-fermentation conditions, different kind of cheeses can be produced. There are a vast selection of cheese consumed worldwide, such as: American, Asiago, Blue cheese, Bocconcini, Brie, Burrata, Camembert, Cheddar, Cheese Curds, Colby, Cotija, Cottage cheese, Cream cheese, Emmental, Feta, Fresh Mozzarella, Gouda, Gruyere, Mascarpone, Mozzarella, Parmesan, Provolone, Romano, Swiss, etc. The present invention aims at producing a cheese substitute comprising casein expressed in a carrot cell slurry, and exhibiting the characteristic aroma, texture and flavor of the above-mentioned cheeses.

As used herein, the term “plant-based yogurt substitute” refers to any consumable product produced by the methods disclosed in the present application using a plant cell slurry expressing transgenic casein proteins, and appearing, tasting, smelling and physicochemically behaving like a conventional yogurt product.

Conventional yogurt is dairy product produced by bacterial fermentation of milk. The bacteria used to make yogurt are known as yogurt cultures. Fermentation of sugars in the milk by these bacteria produces lactic acid, which acts on the milk proteins to confer yogurt its texture and characteristic tart flavor. Cow's milk is the most commonly used milk to make yogurt. Milk from buffalo, goats, ewes, mares, camels, and yaks are also used to produce yogurt. The milk used may be homogenized or not. It may be pasteurized or raw. Each type of milk produces substantially different results in terms of organoleptic properties.

Several kinds of yogurt can be manufactured by implementing different kind of milks and also using different conditions during the manufacturing. The differences are related to flavor and texture. The most important kind of yogurts are: Unstrained, Greek, Goat Milk, Sheep's Milk, Aka Icelandic, Australian, Drinkable, Frozen, Plain, Whole Milk and Low-Fat yogurt.

The present invention aims at producing a yogurt substitute comprising casein expressed in a carrot cell slurry, and exhibiting the characteristic aroma, texture and flavor of the above-mentioned yogurt products.

As used herein, the term “plant-based coffee creamer substitute” refers to any consumable product produced by the methods disclosed in the present application using a plant cell slurry expressing transgenic casein proteins, and appearing, tasting, smelling and physicochemically behaving like a conventional coffee creamer product.

As used herein, the term “casein” refers to a family of proteins commonly found in mammalian milk. These proteins include αS1, αS2, β, and κ. Casein is the main ingredient in cow (bovine) milk, comprising up to about 80% of its protein content. As such, casein is a pivotal component in dairy products, such as cheese, yogurt and ice cream. Nutritionally, casein provides amino acids, as well as calcium and phosphorus. In the context of the present invention, transgenic casein (of an animal origin) is expressed in plant cell culture. The culture is transformed into a powder and then a slurry, which can be the basis for downstream processes for generating dairy substitutes, such as cheese and yogurt. Any type of casein from any known mammalian source (cow, camel, buffalo, sheep, goat etc.) can be expressed in the plant cell culture disclosed in the present invention to generate dairy substitutes.

As used herein, the term “plant cell suspension culture” refers to cells grown in laboratory equipment, under controlled conditions, usually outside their natural environment, isolated from their original tissue. Single cells or small aggregates of cells are allowed to function and multiply in an agitated growth medium, thus forming a suspension of cells. The cells in the suspension can either be derived from a tissue or from another type of culture. In the present application the cells in the suspension culture are preferably carrot cells.

As used herein, the term “slurry” refers to a mixture of solids denser than water suspended in liquid. In the context of the present invention, the slurry comprises disrupted plant cells which are genetically engineered to express casein. Therefore, the slurry contains casein expressed by the cells, and all the intracellular components and content of the plant cells (including fibers, proteins, sugars, pigments, antioxidants etc.)

As used herein, the term “transgenic or recombinant proteins” refers to the expression of proteins through the creation of genetic sequences in a laboratory and introducing them to a system/organism capable of expressing them in mass quantities. In the context of the present invention, transgenic casein of a mammalian origin is amplified in a laboratory and transformed into plant cells (for example, by means of electroporation or agro-infilitration). Subsequently, only cells which successfully absorbed the sequence of the mammalian casein (coupled with a selective gene conferring antibiotic resistance) will be able to survive and multiply and to continue expressing casein.

As used herein after, the term “organoleptic properties” refers to the numerous aspects of foodstuffs, beverages or other substances that create an individual sensory experience such as mouthfeel, taste, sight, smell, texture or touch. The dairy substitute of the present invention exhibits organoleptic properties which are equivalent or similar to conventional dairy products (usually cheese and yogurt). In other words, the product of the present invention may look, smell and taste like non-vegan dairy foodstuffs. Moreover, the products of the present invention have the characteristic textures of non-vegan dairy products in terms of consistency and creaminess.

As used herein after, the term “physicochemical properties” refers to unique physical and chemical properties of a consumable product, which describe among other things, its strength, firmness, tightness, resilience, rheological parameters, moisture content, viscosity, adhesiveness, cohesiveness, hardness, elasticity, springiness, degradation rate, solvation, porosity, surface charge, functional groups etc. The physicochemical properties are responsible for the behavior of the product under different environmental and internal conditions, and they determine for example the product's shelf life, appearance, resistance to stress, interactions with external ingredients, texture and many other aspects.

As used herein after, the term “nutritional values” refers to the measure of essential nutrients, such as fats, proteins, carbohydrates, minerals and vitamins in foodstuffs and beverages. In terms of nutritional values, dairy products, such as cheese and yogurt which are made of animal milk (mainly cows, buffaloes, sheep, goats, camels) are rich in proteins, free amino acids, essential minerals such as calcium, potassium and phosphorus, fatty acids, and some vitamins such as vitamin A, niacin, thiamine, folate B12, C and E. The dairy substitute of the present invention is also enriched with nutritional values as it contains protein (casein), and the beneficial ingredients found in the plant cells in which the casein is expressed. Furthermore, the dairy substitute of the present invention might even comprise enhanced nutritional values compared to conventional dairy products, as it is an industrial product, whose ingredients can be manipulated (meaning that ingredients such as vitamins, minerals and probiotic bacteria can be potentially added to the formulations of the substitute products to fortify their nutritional values).

The present invention provides a method for producing plant-based dairy products, mainly cheese and yogurt, made of recombinant casein protein expressed in plant culture cells. The disclosed system is, in a non-limiting way, a carrot cell suspension culture. The present application discloses the expression and use of bovine alpha S1 (αs1) casein and bovine Kappa (κ) casein, but this is a non-limiting example, and any other type of casein (such as β-casein) can be used to produce the disclosed plant-based dairy substitutes following the description of the present application.

Cheese is a dairy product derived from animal milk and formed by coagulation of casein, the main protein found in milk. The distinct flavor of cheese is determined by the balance between multiple volatile and non-volatile components formed throughout the process of cheese ripening, involving the death and lysis of starter microorganisms, the growth of non-starter lactic acid bacteria and, in certain cheeses, the growth of a secondary microflora which greatly contributes to the flavors and textures of cheese. The biochemical and microbiological changes throughout cheese ripening include primary events and secondary events. The primary events include lactose, lactate and citrate metabolism, lipolysis and metabolism of fatty acids and proteolysis and amino acid catabolism. The latter is described as the formation of large water-insoluble peptides and smaller water-soluble peptides, mainly derived from β- and αs1-casein peptides by the catalytic enzymatic reaction from different sources including the milk itself, and lactic and non-lactic bacteria. In many cheese varieties, αs1-casein hydrolyzes faster than β-casein. In blue veined cheeses both αs1-casein and β-casein completely hydrolyze at the end of the ripening process. The pattern of proteolysis may result in differences among cheese varieties. Those differences could be caused by moisture content, temperature, duration of ripening, cooking temperature and pH at draining. The final products of proteolysis of casein peptides include various organic molecules and compounds, such as keto acids, carboxylic acids, ketones, lactones, esters, alcohols, aldehydes, pyrazines, sulphurous and carbonyl compounds and free amino acids, all of which take part in determining the characteristic flavor of cheese.

In addition to flavor, the proteolysis of casein peptides is highly pivotal for forming the texture of the cheese curd. As water activity decreases, through water binding to the newly formed carboxylic acid and amino groups, substrates such as amino acids become available for secondary catabolic changes (including deamination, decarboxylation, transamination, desulphurization, catabolism of aromatic compounds such as phenylalanine, tyrosine, tryptophan and reactions of amino acids with other compounds). These changes within the cheese matrix enable softening of the cheese, thus affecting its texture.

To date, most types of vegan cheese (cheese substitutes) have failed to capture and mimic the distinctive, stretchy texture and savory flavor characteristic to dairy cheese. The taste of dairy alternatives currently available in the markets is still controversial among consumers, and the main reason for that is the absence of palatable components derived from the proteolysis of casein peptides.

In a preferred embodiment of the present invention, casein peptides are expressed and produced in carrot cell suspension culture serving for the formulation of plant-based dairy substitutes. The dairy substitutes of the present application may be generated using different carrot (Daucus sativus) varieties and cultivars, such as: Snow White, Kurodagosun, Chantenay Red Core, Danvers, Kintoki, Autumn King, Trophy, Amstrong, Flakkee, Nantes, Saint Valery, Brasilia, Emperador, Nerac, Larga Cordobesa, DH1, Nevis F1, Nantaise, Yukon, Amsterdamse Bak, Touchon, Coral, Muscade, and any other lines, varieties or cultivars known in the art, whether they are wild type plants, hybrid plants, progeny of crossing and breeding techniques or genetically engineered plants (transgenic plants or plants whose genome is modified or edited by molecular methods such as CRISPR/Cas). Nowadays, many carrot varieties and cultivars are available in a range of different colors, reflecting a varying spectrum of substances, pigments, vitamins, minerals and antioxidants with scientifically proven beneficial properties. The dairy substitutes of the present invention originate from a slurry of plant cells expressing casein proteins. Said slurry is a pivotal component of the end product (cheese or yogurt substitutes for instance), hence, conferring further valuable nutritional values to the dairy substitutes. For example, if the slurry is made of purple carrot cells expressing casein, then the final product is enriched with minerals and vitamins characteristic to all carrots (potassium, manganese, vitamin C and vitamin A), but is also rich in anthocyanins, which are abundantly found in purple fruits and vegetable.

In yet another preferred embodiment of the present invention, cells from other plant species, which are rich in nutritional values, such as sweet potato (Ipomoea batatas), beetroot (Beta vulgaris), tomato (Solanum lycopersicum), cassava (Manihot esculenta), kohlrabi (Brassica oleracea var. gongylodes), parsley root (Petroselinum crispum), horseradish (Armoracia rusticana), Jackfruit (Artocarpus heterophyllus), rice (Oryza sativa), tobacco (Nicotiana tabacum), potato (Solanum tuberosum), and Anchusa officinalis can be used to express transgenic casein proteins and serve as the platform for the production of the plant-based dairy substitutes of the present invention.

In another preferred embodiment of the present invention, the plant cells (in a suspension culture, in a powder form, in a slurry or as the final plant-based dairy substitute) express at least of the following genetic sequences disclosed in the present application: SEQ ID NO:1, SEQ ID NO:2, SEQ ID NO:3 and SEQ ID NO:4.

In yet another preferred embodiment of the present invention, the plant-based dairy substitutes are characterized by having the distinct organoleptic properties (mainly flavor, odor and texture) associated with conventional dairy products.

In yet another preferred embodiment of the present invention, the plant-based dairy substitutes have nutritional benefits which are not to be found in animal-based dairy products, such as high level of carotenoids, a unique fatty acid pattern and no cholesterol, as the carrot cells serving as the production system are also utilized as nutritional ingredients incorporated into the final plant-based products.

In yet another preferred embodiment of the present invention, the plant cells of the disclosed application expressing casein proteins, can be spray-dried and kept for several weeks without being kept frozen.

In yet another preferred embodiment of the present invention, a slurry of plant cells expressing transgenic casein can be kept for a predetermined period of time at a predetermined temperature (room temperature, refrigerated or frozen for longer periods), and then be used to generate different dairy substitutes, such as cheese, yogurt or coffee creamers. Each production process is different and requires several distinct modifications, but for all products, the same casein-expressing plant cell slurry is utilized.

In yet another preferred embodiment of the present invention, the casein-expressing plant cell slurry is fermented using lactic bacteria. These bacteria can be selected from any lactic bacteria known in the art, such as Streptococcus thermophilus, Lactobacillus helveticus, Lactobacillus bulgaricus, Lactococcus lactis, Lactococcus cremoris, Lactococcus diacetylactis, Lactobacillus acidophilus, Lactobacillus casei, Lactobacillus lactis, Lactobacillus helveticus, Lactobacillus bulgaricus Bifidobacterium etc.

### Example 1

The present application discloses inventive plant-based dairy substitutes (mainly cheese and yogurt analogues), which are also fortified with the naturally occurring nutritional components of the plant cells in which the casein proteins are expressed. Preferably, the plant cells used for the generation of the dairy substitutes of the present invention are carrot cells. The carrot cells comprise an important part of the final product, thus, contributing additional nutritional values and physicochemical properties to the dairy substitutes, as will be described in the following examples.

### Carotenoids

Carrot cells are enriched with numerous materials, such as vitamin A derivates, the carotenoid pigments.

Carotenoids have been shown to have anti-carcinogenic properties in rats and mice, and it also appears to be the case in humans, especially with head and neck cancers (see “Dietary carcinogens and anticarcinogens”, Ames B. N, Science 221: 1256-1264, 1983 and “Carotenoid Intake from Natural Sources and Head and Neck Cancer: A Systematic Review and Meta-analysis of Epidemiological Studies”, Leoncini E. et al, Cancer Epidemiol Biomarkers Prev. 24 (7): 1003-11, 2015). Carotenoids are also beneficial for dermal and ocular health (see “Discovering the link between nutrition and skin aging”, Schagen S K, Dermato-Endocrinology, 4:3, 298-307, 2012).

Although beta-carotene, which is a type of carotenoids, is currently synthetically produced for commercial use, carrot cell cultures offer an environmentally sustainable, green, safe and highly efficient system for producing important plant metabolites.

An additional characteristic of carrot cells as food ingredients is their ability to prolong shelf life of food products due to the presence of both beta-carotene and lycopene. Different studies have shown that the addition of beta-carotene and lycopene to meat and dairy products extends the shelf-life of the product, due to their antioxidant properties, which minimize lipid oxidation and delay surface discoloration. Additional benefits of the disclosed plant-based dairy products are that they are cholesterol-free, since they do not contain any animal fat. Moreover, the daily consumption of carrots has been shown to affect lipid metabolism and reduce cholesterol levels in the blood, mainly due to the fiber content in carrots.

To validate the presence and concentration of carotene by the use of casein-expressing carrot cells, the inventors performed an extraction and determination of beta-carotene in the carrot cell slurry of the present invention.

### Protocol for the Extraction and Determination of α-Carotene, β-Carotene and Lycopene:

1. 5 g of casein-expressing carrot slurry were weighted and ground with a mortar to obtain a paste.

2. 5 ml of cold acetone (4° C.) were added to the tube and maintained at 4° C. for about 15 minutes with occasional manual stirring.

3. The tube was stirred with vortex at high speed for 10 minutes and then centrifuge at 1370×g for 10 minutes.

4. The supernatant was collected in a separate tube, and 5 ml of cold acetone was added again to the precipitate.

5. Step 4 was repeated and both supernatants were collected into the same tube.

6. The absorbance at 449 nm was measured by using UV-Vis spectrophotometer.

For the determination of the α-carotene, β-carotene and lycopene concentrations, the inventors used the equations reported elsewhere for carrot tissue samples:

$${C}_{α - carotene} ( \frac{mg}{Lt} ) = 7.04 {A}_{443 n m} - 10.11 {A}_{492 n m} + 2.06 {A}_{505 n m} \\ {C}_{β - c a r o t e n e} ( \frac{mg}{Lt} ) = - 4.24 {A}_{443 n m} + 13.80 {A}_{492 n m} - 6.70 {A}_{505 n m} \\ {C}_{Lycopene} ( \frac{mg}{Lt} ) = 0.21 {A}_{443 n m} - 1.63 {A}_{492} n m + 4.18 {A}_{505 n m}$$

Where C_{α-carotene}, C_{β-carotene}, C_{Lycopene} are respectively the concentration of α-carotene, β-carotene and lycopene in mg per liter, and A_{443 nm}, A_{492 nm}, A_{505 nm} are respectively the absorbance at 443 nm, 492 nm and 505 nm.

After analyzing the samples in triplicates, the mean absorbance values obtained were as described in Table 1:

TABLE 1
Mean absorbance values for the determination
of α-carotene, β-carotene and lycopene

| | A_{443 nm} | A_{492 nm} | A_{505 nm} |
|---|---|---|---|
| Carrot cell extract | 3.342 | 2.842 | 2.727 |
| in acetone | | | |

After applying the absorbance values of Table 1 in the above-referenced equations, the following concentrations were calculated:

C_{α-carotene}=0.413 mg/L

C_{β-carotene}=6.448 mg/L

C_{Lycopene}=7.471 mg/L

Taking into count that the wet weight of one sample of carrot cell slurry was 5 gr, the approximate contents of α-carotene, β-carotene and lycopene per gr of wet carrot cell slurry are 0.826 μg, 12.89 μg and 14.94 μg, respectively.

These values indicate that the carrot cell slurry disclosed in the present application and used for the production of various dairy substitutes contains high levels of carotenoids. These carotenoids are an added nutritional value to the plant-based dairy substitutes, as conventional dairy products do not naturally contain carotenoids.

### Fatty Acids Profile

Overconsumption of saturated fatty acids (SFA) is a main concern in many developed countries, while at the same time most of developing countries suffer from underconsumption of polyunsaturated fatty acids (PUFA), which are considered more beneficial to human health. According to the Dietary Guidelines for Americans (2015-2020), daily intake of fats should not exceed 20-35% of total acquired energy. Furthermore, not more than 10% of energy should be obtained in the form of saturated fatty acids. Polyunsaturated fatty acids consumption is recommended to constitute 5-10% energy from n-6 and 0.6-1.2% energy from n-3, with not less than 0.5% energy from α-linolenic acid (ALA) and 250 mg per day of eicosapentaenoic acid (EPA) and docosahexaenoic acid (DHA). In turn, most recommended daily intake of conjugated linoleic acid (CLA) for adults is 0.8 gram per day. Generally, the high contribution of animal fat in human diets linked with high cholesterol intake is believed to be associated with the occurrence of diet-related diseases such as coronary diseases and metabolic syndromes.

Glycerolipids are major components of the membrane architecture in plant cells. These acyl lipids are diester of fatty acids (FAs) and glycerol, and the FA moieties can be either saturated or unsaturated. In higher plants, the main species of FAs are 16C and 18C, representing respectively about 30% and 70% of total FAs. These FAs are present with various saturation levels, generally displaying none (16:0, 18:0) to three (16:3, 18:3) double bonds for the main species. In the case of carrot cell culture, the FA profile is ranging as follows: linoleic acid (53-69%), palmitic (27,32%), linolenic (4-10%), oleic (ca. 6%), and stearic (0-1.8%) acids.

### Casein and Calcium

Calcium is a very important nutrient for the human body, since it is the main component of bones and teeth, and has many important physiological functions. An insufficient absorption of calcium in the intestine is one of the main causes of diseases such as osteoporosis. However, calcium is difficult to absorb from the diet directly due to the precipitation of insoluble calcium salts in basic environments such as in the small intestine. Disease-related organ malfunction, physiological ageing processes, and other diseases are known to be associated with the disruption of calcium homeostasis.

It has been proved that caseins release bioactive peptides containing phosphate (Serp-Serp-Serp-Serp-Glu-Glu) by the action of proteolytic enzymes present at the intestine. These bioactive peptides are known as calcium-promoting factor, because they limit the precipitation of calcium in the small intestine, thus playing a beneficial role in calcium absorption and bone mineralization. Furthermore, it has been demonstrated the bioactive peptides derived from caseins may directly affect osteoblast-like cell growth, calcium uptake, and ultimately calcium deposition in the extracellular matrix. Indeed, this peptide-mediated increase in the bioavailable fraction of the mineral at the intestinal level could implement the availability of calcium for bone, potentially resulting in the enhancement of bone calcium content and the modulation of the cellular activity.

Calcium is a mineral present in different foodstuffs of plant origin such as soy, beans, peas, lentils, seaweed, and certain nuts among others. However, the percentage of intestinal absorption is lower compared to the calcium consumed in dairy foods. The main reason is that plant sources lack phosphorylated casein-derived peptides. The development of a viable plant-based source of casein for the incorporation and production of dairy products represents a significant improvement in the nutritional quality of these products.

### Example 2

### Experimental Design for the Expression of Bovine Caseins in Carrot Cell Suspension Cultures.

### A. Development of Fast-Growing Carrot Cell Lines for the Accumulation of High Amount of Biomass.

Different varieties and cultivars of carrot (Daucus carota) where assayed for the establishment of fast growing in vitro cultured cell lines. The dairy substitutes of the present application may be generated using different carrot varieties, for instance: Snow White, Kurodagosun, Chantenay Red Core, Danvers, Kintoki, Autumn King, Trophy, Amstrong, Flakkee, Nantes, Saint Valery, Brasilia, Emperador, Nerac, Larga Cordobesa, DH1, Nevis F1, Nantaise, Yukon, Amsterdamse Bak, Touchon, Coral, Muscade. And any other lines, varieties or cultivars known in the art.

Carrot seeds were soaked in water overnight at 4° C. and then surface sterilized by dipping in 70% ethanol for 1 min, treated for 5 min in a 20% bleach solution, and then rinsed 5 times in sterile distilled water. The seeds were germinated on half-strength Murashige and Skoog (MS) medium with 0.25% sucrose and 0.8% agar (pH 5.8) at 26° C. and under cool-white fluorescent lights (450 μmol m⁻² s⁻¹, 16 h day/8 h night).

When the length of the hypocotyls was around 1 cm long, seedlings were removed, petioles and hypocotyls were excised, and 2-3 mm length segments were used for the induction of calli formation.

Explants were placed on calli induction plates (3.2 g/L Gamborg B5 basal medium, 0.5 g/L MES (2-(N-morfolino) ethanesulfonic acid), 2% sucrose, 0.7% agar, pH 5.7, and supplemented with 1 mg/L 2,4-dichlorophenoxyacetic acid (2,4-D), 0.1 mg/L kinetin, after sterilization by autoclave). Plates were incubated at 26° C. and under cool-white fluorescent lights (450 μmol m⁻² s⁻¹, 16 h day/8 h night) and calli formation monitored during 3-4 weeks.

Fresh calli of pale-yellow color and friable (approximately 0.3-0.5 g) were removed from induction plates and used as inoculum to start liquid cultures in 50 mL Erlenmeyers containing 10 mL of carrot cell culture medium (3.2 g/L Gamborg B5 basal medium, 0.5 g/L MES (2-(N-morfolino) ethanesulfonic acid), 2% sucrose, pH 5.7, and supplemented with 1 mg/L 2,4-dichlorophenoxyacetic acid (2,4-D), 0.1 mg/L kinetin). Cultures were incubated with agitation at 130 rpm, 26° C. and under cool-white fluorescent lights (450 μmol m⁻² s⁻¹, 16 h day/8 h night). Initiated cell lines were initially self-cultured every 7-10 days using 30% of inoculum. Once cell lines tolerate self-culture every 7 days, inoculum was sequentially reduced to adapt cells to fast growth. If the carrot variety used for the disclosed system is for example, Snow white, then cell lines are maintained by self-culturing using 15% inoculum every 7 days in induction medium.

### B. Obtaining Plasmid Constructs for the Expression of Bovine Caseins (CSNs) in Carrot Cells.

To express bovine CSNs in the system of the present application, the coding region of B. taurus α_{S1}-CSN (NM_181029, SEQ ID NO:1) and κ-CSN (BC102120, SEQ ID NO:2) sequences were codon optimized for their expression in carrot (Daucus carota) (SEQ ID NO:3 and 4 respectively). In their native hosts, caseins are synthesized at the endoplasmic reticulum (ER), packaged into Golgi derived vesicles, and secreted by exocytosis to the milk. To evaluate CSNs accumulation into plant cells, these proteins were expressed as cytoplasmic soluble proteins and for that their signal peptides (highlighted in bold on SEQ ID NO: 1 to 4) where removed. These sequences were ordered as synthetic genes cloned into pDonr221™ plasmid thus obtaining pDONR-α_{S1}-CSN_{wt}, pDONR-α_{S1}-CSN_{DC}, pDONR-κ-CSN_{wt} and pDONR-κ-CSN_{DC}. Using the Gateway™ LR Clonase™ Enzyme mix (Thermo Fisher Scientific), the CSNs sequences were transferred to the following plant binary vectors: (i) pB7WGF2, obtaining pB7-GFP:α_{S1}-CSN_{wt}, pB7-GFP:α_{S1}-CSN_{DC}, pB7-GFP:κ-CSN_{wt} and pB7-GFP:κ-CSN_{DC}, vectors for the expression of both WT and carrot optimized versions of the CSNs fused from their amino terminal part to Green fluorescent protein (GFP); and (ii) pK2GW7 obtaining pK2-α_{S1}-CSN_{wt}, pK2-α_{S1}-CSN_{DC}, pK2-κ-CSN_{wt} and pK2-κ-CSN_{DC}, vectors made for the expression of WT and carrot optimized untagged bovine CSNs in carrots cells. Both constructs express the gene of interest under the control of the CaMV 35S constitutive promoter and 35 terminator sequences. The plasmids were then transferred by electroporation to Agrobacterium tumefaciens GV3101 strain electrocompetent cells and transformants were selected by incubation on selective antibiotics (rifampicin [25 μg/mL], gentamicin [100 μg/mL] and spectinomycin [50 μg/mL] containing Luria Broth agar plates during 48 hours at 28° C.

C. Transient Expression of Bovine αS1-CSN and κ-CSN in Nicotiana benthamiana Leaves

Transient expression of bovine caseins was performed by agro-infiltration into N. benthamiana leaves. Selected A. tumefaciens harboring both GFP tagged and untagged CSNs constructs where grown under proper selective antibiotics on LB medium overnight, at 28° C. with 180 rpm agitation. Cultures were harvested by centrifugation, resuspended in water to a final OD_{600 nm} of 0.3 and infiltrated into the abaxial side of the leaf using a syringe without needle. Leaves agroinfiltrated for the expression of GFP tagged CSNs were observed under epifluorescence microscope at 2-3 days post agroinfiltration (dpai). All GFP tagged CSNs (WT and carrot optimized) accumulate at the cytoplasm and nucleus. Observation at cortical planes of the cells indicate that the proteins remain soluble, without accumulating at any obvious subcellular structure and follows the acto-myosin driven cytoplasmic streaming. The expression, size, and integrity of the caseins were confirmed by western blot. For that, total protein extraction was performed and detected using both anti-GFP monoclonal antibody (3H9, Chromotek) and anti-milk proteins antiserum raised in rabbit as primary antibodies. Horseradish Peroxidase (HRP) conjugated anti Rat (7077S, CST) and HRP conjugated anti-rabbit (Goat anti-Rabbit IgG (H+L), Invitrogen) were respectively used as secondary antibodies. Chemiluminescent reagent was used for developing. Similarly, untagged CSNs was analyzed by W. blot using anti-milk serum, confirming its expression and accumulation at 4 and 7 dpai.

### D. Transformation of Carrot Cell Lines for the Constitutive Expression of Bovine αS1-CSN and κ-CSN.

Carrot cell were transformed by co-culture with A. tumefaciens strain GV3101 harboring pK2-α_{S1}-CSN_{DC} and pK2-κ-CSN_{DC} plasmids. Agrobacteria was grown over night on LB medium supplemented with proper selective antibiotics (rifampicin, gentamicin and spectinomycin at 25, 100 and 50 μg/μl respectively), harvested by centrifugation at 5000 g for 10 min and resuspended to an OD_{600 nm}=0.2 in carrot induction medium supplemented with 200 μM acetosyringone and incubated for 2 h at 22° C. 10 mL of exponentially growing carrot cells were vacuum filtered onto a filter paper disc until liquid was removed. The disc was inoculated with 500 μl of induced agrobacteria. The mixture was then co-cultured on plates containing plant cells medium without antibiotics, at 25° C. and on the dark. After 3 days, co-cultured cells were harvested and transferred to 15 mL sterile tubes, washed 3 times with 10 mL of plant cells medium containing cefotaxime (250 μg/mL) and kanamycin (100 μg/mL). For every wash, cells were centrifugated at 400 g for 5 min and supernatant discarded. Resulting pellets at each wash were resuspended by gently agitation. After the last wash, cells were resuspended in 10 mL medium, and 1 mL was poured on agar plates containing carrot cells medium supplemented with cefotaxime (250 μg/mL) to eliminate remaining bacteria, and kanamycin (100 μg/μl) to select transgenic resistant cell lines.

Plates were incubated in the dark at 25° C. for 3-5 weeks and monitored periodically for the presence of calli. Calli were harvested and cultured on plates containing kanamycin (100 μg/mL). Approximately 0.3-0.5 g of calli belonging to each transformation event were used to initiate liquid cultures in 50 mL Erlenmeyers containing 10 mL of carrot cells medium supplemented with kanamycin (100 μg/mL). Transgenic cell lines were self-cultured every 7 days under selective conditions and assayed for the accumulation of casein. Cell lines accumulating high levels of the transgenic protein were selected and used for subsequent protein characterization.

### E. Fermentation Batch

The resistant calli were cultured in a Murashige and Skoog liquid medium supplemented with adenine (5-11 μM) and 2,4-D (0.5-2 μM). The suspensions were sub-cultured every ten days in the same medium. 2 ml of packed cell volume filtered through steel screens of 500 and 100 μm pore size were inoculated into 100 ml of fresh medium. All cultures were kept in Erlenmeyer flasks maintained at 26° C. on an orbital shaker (100 rpm) under a photoperiod of 16 hours. The fluorescent source (maximum fluence rate 45 μmol m-2s-1) consisted of 58 W white daylight tubes.

For the batch fermentation a 6-liter glass vessel bioreactor with a working volume of 4.0 L was used. Temperature was maintained at 27° C. using a water jacket. The bioreactor was equipped with oxygen and pH probes to monitor their respective levels. Mixing was carried out using four blade impellers at about 50-100 rpm during the growth phase. The aeration rate was achieved using a compressor, and it was maintained constant at 100-200 ml/min for the proliferation phase. The bioreactor was loaded with about 5-9 g (fresh weight) of an inoculum of aggregate cells whose size was between 100 and 500 μm. The growth medium was identical to that used for the cultures in the Erlenmeyer flasks. The reaction was conducted under a photoperiod of 16 hours (the maximum fluence rate was 25 μmol m⁻² s⁻¹). During the growth phase, silicon was added to avoid formation of foam on the surface of the suspension. For the determination of the growth curve, samples from the culture were taken, and a known volume was filtered through a GF/A filter (Whatman) under a reduced pressure. The recovered cells were weighed, and then were dried for 24 h at 80° C. to determine the dry weight. The growth rate (u) was calculated during exponential growth as the slope of a linear regression of the In (dry weight) versus time. The doubling time (Td) was based on the growth rate where Td=0.542/L. The results show a u=0.477 and a Td=1.3 days.

### Example 3

Following the protein expression process described in the above example, all cellular materials from the bioreactor are harvested by vacuum filtration. Subsequently, the wet carrot cells were resuspended in an aqueous buffer that adjusts the pH to 7.2. In addition, the buffer contains EDTA (Ethylenediaminetetraacetic acid, a chelating agent), ascorbic acid (antioxidant), polyvinyl pyrrolidone (polyphenol scavenger), Triton X-100 (detergent) and maltodextrin (carrier). The buffer volume is high enough to achieve a dry matter content of 45%.

First, the maltodextrin and all the other additives are dissolved using a shear disperser (Ultra Turrax 50, IKA, Germany) for 10 min at 7000 rpm at 25° C.

The carrot cell suspension was spray-dried in a pilot scale spray dryer (Anhydro Lab S1, Denmark) equipped with a two-fluid nozzle which was installed for a co-current spray drying process. To produce spray-dried particles with various size ranges, the total dry matter of the suspension is 45% (w/w), as well as the atomizing pressure of the spray nozzle of 2 bar(g) were adjusted according to a 22 factorial design with a center point at 1.5 bar(g) atomizing pressure and 45% (w/w) dm in the suspension. The inlet air temperature was set at 195° C. In order to retain the outlet air temperature at 80° C. during each trial, the feed rate was adjusted by the speed of the attached peristaltic feed pump. 1,500 g of suspension was dried in each run at a feed flow rate of 30-42 g/min. Samples were collected from the sampling container, which is installed after the cyclone. The powder yield was determined as the ratio of collected powder from the sampling container to the theoretical total dry matter of the atomized suspension. The highest yield reached at the trials was 88%. The powder product can be stored at about 25° C. up to six months, maintaining its functionality.

Unlike other recombinant food protein expression platforms (such as yeasts), plant cells have a thick wall composed of cellulose fibers that allow them to act as a beneficial encapsulation system. This cell wall confers resistance against external conditions of physicochemical, enzymatic and oxidative stresses.

Carrot cells can be dried by spray drying allowing a longer life as a food ingredient. It was previously reported that a spray-dried product of carrot cells contained up to 80% of the carotenoid content even after 12 weeks of storage at 35° C.

The present application aims at obtaining ingredients (carrot cells that contain transgenic alpha S1-casein or kappa-casein for the formation of dairy substitutes) that can be dried by spray-drying while maintaining the viability of the expressed transgenic proteins, and simultaneously the ingredients can be stored and transported at 25° C., without refrigeration. This feature is significantly advantageous compared to yeast-based platforms, which do not support spray-drying and must be frozen and kept in this state during storage. Commercially, keeping dried, frozen yeast-based products is highly unprofitable, since these products must be consumed almost immediately after the end of the production process. On the other hand, the transport of frozen food ingredients is also not a commercially viable technique.

Due to the absence of a cell wall, yeasts cannot protect the proteins found inside them, from thermal and oxidative stresses caused by spray drying. Therefore, companies that use this expression platforms must break the cells once the protein expression process is finished, and follow one of the following routes:

Directly applying the yeast protein extract to the food formulation. This should be done immediately after partial purification.

Alternatively, freezing the protein extract could be carried out, which should prolong the ingredients' shelf life for a few weeks. However, the conservation of large volumes in a frozen form is a highly expensive industrial practice and therefore, not recommended.

Lastly, the protein extract can be lyophilized, which allows the manufacturers to have a powder product lasting for several weeks. However, lyophilization is a very expensive process reserved practically only for the pharmaceutical industry.

Hence, companies that use yeasts as an expression platform, inevitably need to have fermentation capacities in each country they want to market, limiting their capacity for commercial expansion.

Unlike yeast-based platform, plant cells have a cell wall, which is a structure characterized by a high lignin content, especially carrot cells. The fiber content allows carrot cells to be excellent protein carriers. Many investigations have been carried out to use carrot cells for oral delivery of therapeutic proteins, due to their ability to protect the proteins found inside them from oxidative and enzymatic stress resulted by the stomach and digestive tract (see “Protein delivery into plant cells: toward in vivo structural biology.” Cesyen Cedenyo et al. Front Plant Sci. 2017; 8: 519, 2017). Similarly, carrot cells can protect their inner protein content from the oxidative damage caused by the spray drying process.

Obtaining recombinant proteins in plant cells capable of being spray dried while still preserving the integrity of the proteins, allows exportation of foodstuffs and ingredients from the country of production to any part of the world.

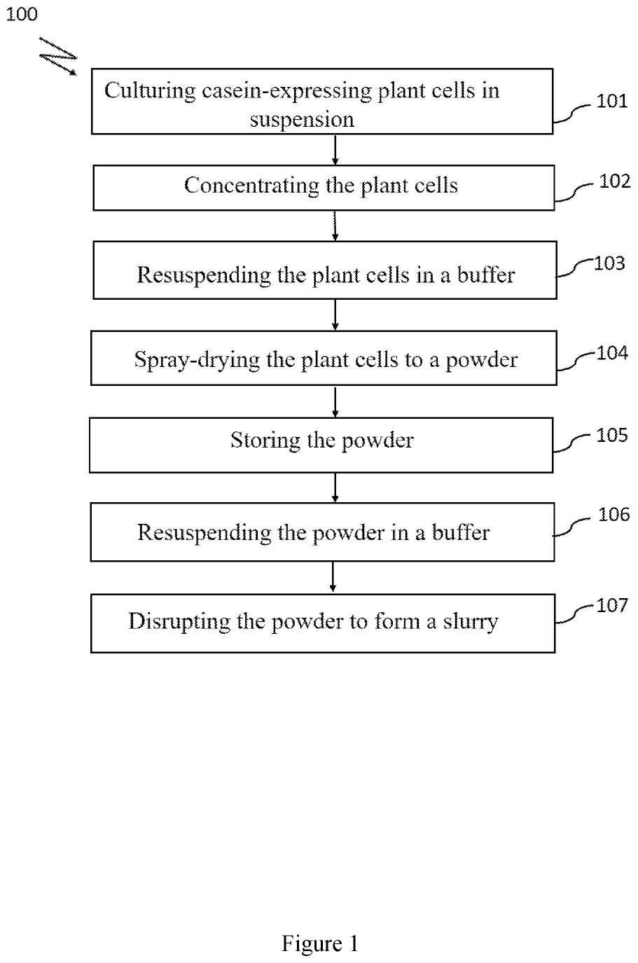

Reference is now made to FIG. 1. schematically depicting the method for preparing the slurry which is used for the production of the plant-based dairy products disclosed in the present application. First, plant cells (preferably carrot cell) which are genetically manipulated to express casein (such as bovine αs1-casein and κ-casein), are suspended in a culture (101), with optimal conditions allowing them to multiply. Once a sufficient amount of plant cells is obtained in the culture, the suspension is concentrated by any concentration mean known in the art, for example, vacuum filtration or using a membrane (102). Then, the plant cells are resuspended in a buffer solution (103) and spray-dried (104) until the formation of a fine powder. Subsequently, the powder is stored at a suitable temperature (for carrot cells at about 25° C.) (105) till further use. The following steps are resuspension of the powder in a buffer solution (106), and disruption of the plant cells by means such as homogenization (107) to release ingredients from inside the cells. The carrot cell slurry containing transgenic casein can now be applied to produce the plant-based dairy products of the present invention.

### Example 4

The plant-based dairy substitute of the present invention can be manufactured in several different ways with slight modifications during the preparation process to produce different products, such as cheese substitute, yogurt substitute, cream substitute, ice cream substitute, coffee creamer substitute and custard substitute. The following examples (examples 4-7) refer to the production of a plant-based cheese substitute.

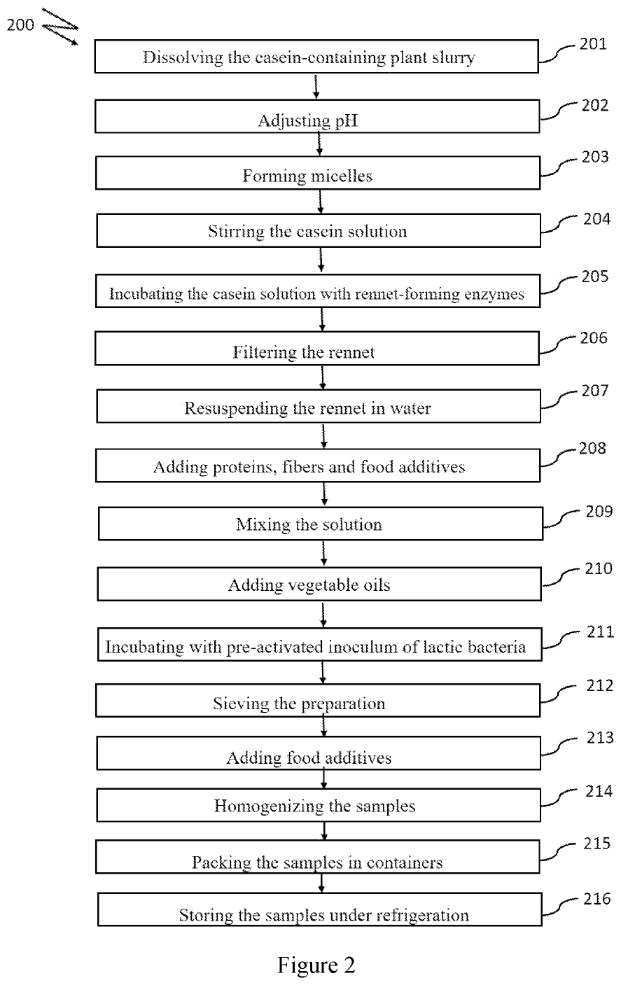

Reference in now made to FIG. 2 depicting the method for the downstream production of the plant-based cheese product disclosed in the present application (after the slurry is generated as described in Example 2 and FIG. 1). The method (200) commences after transgenic casein proteins (such as αs1-casein and K-casein) are expressed and purified from the plant cell suspension culture. Initially, the slurry containing transgenic caseins is dissolved (201) until reaching 0.1-10% of αs1-casein and 0.1-2% of κ-casein, in the corresponding volume of water. The pH of the solution is adjusted to 6.7 by adding suitable acids or bases (202). Then casein micelles are formed (203) by adding about 10% v/v of CaCl₂ solution (0.2 M) and about 5% v/v of K₂HPO₄, while the casein solution is kept under strong stirring at room temperature. When the addition of chemical is completed, the casein solution is kept under strong stirring for additional 15 minutes (204). If necessary, the pH is re-adjusted to 6.7 using suitable acids or bases. For rennet formation, the solution of casein is incubated with enzymes, such as chymosin (20 IMCU/Lt) at about 35° C. for about 15 minutes (205). The rennet is then filtrated (206) and resuspended in water at 0.5-3.5% of concentration (207). Then, various ingredients are added to the rennet, such as fibers, proteins, stabilizers or other food additives (208). Such ingredients can be in a non-limiting way: (i) plant fibers (about 0.1-0.5%) originating from sources such as wheat, oatmeal, bran, root vegetables or legumes; (ii) starch (about 2-4%) such as potato starch or corn starch; (iii) plant-based proteins (about 3-5%), such as pea protein, chickpea proteins, quinoa proteins, lentil proteins, lupine proteins, bean proteins, flaxseed proteins etc.; and (iv) yeast extract (about 1%) or free amino acids or peptides. The solution is maintained in high shear mixing until complete homogenization (209). In terms of fats, plant-based oils are also added to the solution (210) while constantly maintaining the high shear mixing. Any suitable vegetable oil can be added to the solution separately or as a combination of oils, for instance, canola oil (about 3-5%), coconut oil (about 7-14%), palm oil (about 2-5%), olive oil (about 3-6%), sunflower oil (about 4-9%), grapeseed oil (about 1-6%), corn oil (about 3-5%), cottonseed oil (about 1-6%), peanut oil (about 2-5%), sesame oil (about 1-4%), soybean oil etc. Subsequently, the preparation is incubated at about 40±2° C., with a pre-activated inoculum of lactic bacteria strains (such as Streptococcus thermophilus, Lactobacillus helveticus and Lactobacillus bulgaricus) (211) until reaching a pH of 4.5±0.1 (2 hours approximately). Moreover, 1 g of CaCl₂ can be added to obtain a proper floc, and the sample is allowed to stand at 65-75° C., approximately for about 60 min. At this point, the appearance of white clouds on a yellow serum is observed. The preparation is then poured onto a sieve covered with cheesecloth for the drainage of whey (212), which is collected in a graduated cylinder. The curd is weighed and additives, such as NaCl (1.5 gr/100 gr) and Annatto extract (1 mg/100 gr) are added directly to the mass (213). The samples are then homogenized until a creamy texture is obtained (214). Next, the samples are transferred into sanitized/sterilized containers of 100 g (215), weighed and stored in a frigorific chamber (216) with controlled temperature and humidity.

### Example 5

### Physicochemical Analysis and Yield Evaluation of the Plant-Based Cheese Substitute of the Present Application

pH of the plant-based cheese substitute was measured using a digital pH-meter for viscoplastic substances. Titratable acidity was determined with 0.1 mol/L NaOH, expressed as lactic acid. The total protein content was calculated by determination of total nitrogen by the Kjeldahl method using a digestion block and a semiautomatic Kjeldahl Distiller. The fat content was measured by the Röse-Gottlieb method (AOAC 15029). Total solids were determined by weight difference, drying in an oven at 70±1° C. for 24 h (AOAC 15016). The determination of syneresis was carried out, after 24 hours of storage under cold conditions. The gels were stirred for 60 seconds on a platform and centrifuged for 20 minutes at 5000 rpm in an ultracentrifuge at 4° C. using Sorval WX 480 centrifuge (Thermo Fisher). Syneresis, S (g/100 g) was calculated as mass of serum that separated from the gel due to centrifugation, related to the total mass of gel that was centrifuged, as depicted in the following equation:

$$S = \frac{m ( serum )}{m ( gel )} \times 100$$

The actual yield (Ya) of the cheese substitute production was calculated as depicted in the following equation:

$$Ya = \frac{weight of product}{weight of microemulsion} \times 100$$

The results of physicochemical analysis of the control sample (a plant-based cheese substitute manufactured according to the embodiments and examples of the present disclosure, with a carrot cell slurry which is not transgenic and does not express casein) and the samples with the incorporation of transgenic αs1-casein and kappa-casein are shown in the following Table 2.

TABLE 2
Physicochemical analysis of the plant-based cheese product of the present invention

| Experimental | | | Fat | Total Protein | | Actual |
|---|---|---|---|---|---|---|
| Parameter | pH | Acidity | (g %) | (g %) | Moisture | Yield |
| Control | 5.77 ± 0.08 | 0.99 ± 0.16 | 3.40 ± 0.33 | 3.66 ± 0.08 | 68.01 ± 0.10 | 12.40 ± 2.15 |
| Transgenic αs1- | 5.62 ± 0.11 | 0.97 ± 0.12 | 3.75 ± 0.41 | 10.26 ± 0.05 | 70.80 ± 0.70 | 20.03 ± 3.50 |
| casein and κ-casein | | | | | | |

The moisture values show a slight increase with the addition of transgenic casein proteins; this may be due to the water retention by the casein micelles. The protein content of the samples increased in the samples containing transgenic caseins compared to control sample which did not contain casein proteins. There was an average increase of 6.6% of protein in the plant-based cheese substitute. These values are in agreement with the actual yield obtained. In this sense, the yield for this type of plant-based cheese products is higher than other cheeses due to the high moisture content. Furthermore, the high performance could be related with water retention capacity of casein micelles.

### Example 6

### Determination of Rheological Parameters of the Plant-Based Cheese Substitute of the Present Application:

The viscoelastic behavior of the cheese substitute was determined by oscillatory tests evaluating the elastic modulus G′ and the viscous modulus G″. The tangent of phase shift or phase angle (tan δ=G″/G′) was also evaluated. All measurements were made in duplicates at 20° C. in an oscillatory rheometer equipped with a temperature control sensor using parallel 35 mm plates (PP 35/S), with a 1.5 mm gap between plates. The surfaces of both upper and lower plates were serrated to remove the possibility of slip at the ends.

First, stress sweep tests (0.01-1000 Pa) were performed to determine the linear viscoelastic range at a constant frequency (1 Hz-6 28 rad s-1). In addition, the apparent viscosity was measured with a rotational viscometer (with a rotor 2) which has a shear rate of 3750 s-1 at 15±0.5° C.

### Rheological Parameters:

The data obtained through this type of dynamic (or oscillatory) measurements are the contributions to the internal structure of the sample from the elastic and viscous portions of flow, G′ and G″ (Pa), respectively. The measurements in the linear viscoelastic region involve probing the structure of the sample in a non-destructive manner. For the samples containing transgenic caseins it was found that the value of G′ was above G″. In other words, the sample has the capacity to store energy and it is able to return, to some extent, to its initial configuration before a mechanical force was applied on it. The sample behaves as an elastic solid, due to the predominating elastic components. This may be due to the microstructure provided by casein micelles allowing viscoelastic behavior. In contrast, samples without casein (control samples) showed a G″ value which is higher than G′, meaning that the applied force was higher, the microstructure collapses and the mechanical energy given to the material was dissipated.

### Example 7

### Texture Profile Analysis of the Plant-Based Cheese Substitutes of the Present Application

Texture is an important indicator for evaluating cheese quality and functional characteristics, which are also commonly used to differentiate many varieties of cheese. Cheese texture is considered to be a determinant of the overall opinion and preference of the consumers. The major approaches for analyzing cheese texture are sensory evaluation and instrumental measurements. The former approach is however, time-consuming and requires extensive training of panelists; thus, the latter approach is often chosen for routine analysis of cheese texture. Texture profile analysis (TPA) works effectively for analyzing and predicting sensory attributes of cheese. Numerous studies confirmed that the results of instrumental TPA correlated well with sensory evaluation data of cheese texture. (M. A. Drake, P. D. Gerard, V. D. Truong, and C. R. Daubert, “Relationship between instrumental and sensory measurements of cheese texture,” Journal of Texture Studies, vol. 30, no. 4, pp. 451-476, 1999) (R. Di Monaco, S. Cavella, and P. Masi, “Predicting sensory cohesiveness, hardness and springiness of solid foods from instrumental measurements,” Journal of Texture Studies, vol. 39, no. 2, pp. 129-149, 2008.)

Cheese rheology is an important tool to study and identify the textural and structural properties. It deals with deformation of the sample by employing different kinds of instruments. Results of the small and large deformation tests are interpreted to understand the effect of composition, process modification, storage etc. With the introduction of advanced instrumentation texture profile analysis (TPA), small amplitude oscillatory shear test-dynamic stress rheology and stress relaxation tests have been employed routinely in cheese research.

The TPA curve recorded by the GF Texturometer depicts a specimen's force-time relationship (labeled force-deformation relationship) in a double uniaxial compressive test performed with two parallel plates at a constant linear displacement (deformation) rate. In the first cycle, the specimen continues to be compressed after yielding to a set displacement, and then its remnants were compressed again after the crosshead had been (rapidly) withdrawn. Although the curves are recorded under very different test conditions from those in the GF Texturometer, their features are described in very similar mechanical/textural terms, implying that they are the same objective measures of the corresponding material properties.

Instrumental texture profile analysis (TPA) of the control samples and the plant-based cheese substitute containing the transgenic casein proteins, was performed on a TAXT2i Texture Analyzer (Stable Micro Systems) using a load cell of 25 kg and a disc-shaped probe (45 mm of diameter). Testing was carried out after the samples had been equilibrated to a standard temperature of 12±1° C. Two consecutive compressions automatically performed at a test speed of 0.5 mm s⁻¹ and compression ratio of 30%. The waiting time between one cycle and another was set at 5 seconds. From the force curve versus time, the following mechanical properties were determined: hardness (N), adhesiveness (N), springiness, cohesiveness and resilience.

The following definitions for the above-mentioned physicochemical parameters are elaborated:

Hardness: The resistance of metal to penetration by a pressed hard metal ball (Brinell), a pointed diamond cone (Rockwell) or pyramid (Vickers), determined by the indentation size after the load removal (plastic deformation). It is expressed in the Mohs scale where diamond has the value of 10, the hardest, and talc 1, the softest.

Adhesiveness: Adhesion of materials to other materials' surfaces (e.g., glues) is usually determined by a peel test. It refers to the strength of the physical attraction between different materials (unlike cohesion that refers to the attractive forces within the same material which keep it together).

Cohesiveness: Cohesion in soil mechanics and powder technology is defined at the shear stress under zero normal stress. It has stress (pressure) dimensions and units and is not a ratio of areas.

Resilience: It is the ratio of areas from the first point of inversion of the probe to the crossing of the x-axis and the area produced from the first compression cycle. Resilience is a measure of how well a product can regain its original shape and size.

The data measured by the texture analyzer are presented in the following Table 3.

TABLE 3
Texture profile analysis of the plant-based cheese substitute of the present invention

| Sample | Hardness (N) | Cohesiveness | Springiness | Adhesiveness (N) | Resilience |
|---|---|---|---|---|---|
| Control | 5.10 ± 0.50 | 0.50 ± 0.02 | 0.30 ± 0.10 | −2.40 ± 0.30 | 0.08 ± 0.01 |
| Transgenic αs1- | 18.10 ± 1.00 | 0.48 ± 0.03 | 0.90 ± 0.10 | −7.10 ± 1.10 | 0.10 ± 0.01 |
| casein and κ-casein | | | | | |

The application of analysis of variance revealed no significant differences among the tested samples in terms of cohesiveness and resilience. However, there were statistically significant differences for the springiness, adhesiveness and hardness parameters with the latter showing the most pronounced difference among the samples (18.10 vs. 5.10). Hardness of the samples increased due to the addition of transgenic casein proteins to the product. This result should be related to the elastic modulus G′ (explained in Example 6), since the higher the G′ value, the greater the energy required to deform it, suggesting that the material is harder. Regarding springiness and adhesiveness, for both parameters larger values were measured in the samples containing casein micelles (0.90 vs. 0.30 and −7.10 vs. −2.40, respectively). With respect to resilience (the ability of a sample to restore its original configuration after being applied a force responsible for the deformation), results show that the presence of casein micelles enhance the elasticity values.

### Example 8

As mentioned above, the plant-based dairy substitute of the present invention can manifest as several different products, one of which is a yogurt substitute. The following examples (8-11) describe the method of producing said plant-based yogurt substitute and its organoleptic and physicochemical properties. The plant system (plant cell cultures) expressing transgenic casein and the production of a plant slurry and powder used for the generation of the yogurt substitute are disclosed in examples 2-3 and FIG. 1 of the present application.

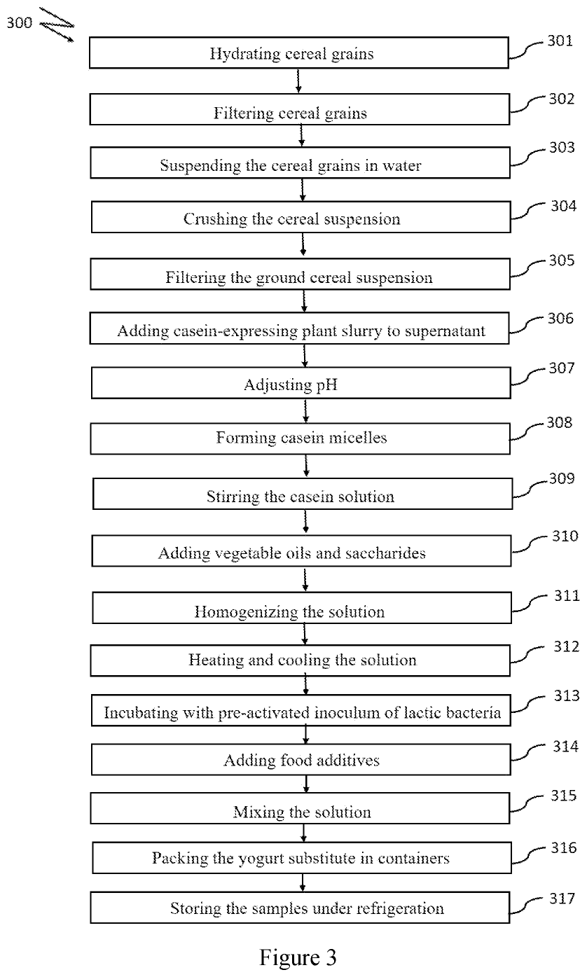

Reference in now made to FIG. 3 depicting the method (300) for the downstream production of the plant-based yogurt substitute disclosed in the present application. The method (300) commences after casein proteins (such as, bovine αs1-casein and K-casein) are expressed and purified from the plant cell suspension culture, as describe in examples 2-3. Initially, cereal grains (for example oat, rye, spelt and triticale) are added to water in about 24% concentration and left to hydrate at room temperature for about 4 hours (301). The hydrated cereal grains are filtered and the supernatant is discarded (302). The filtered solid is resuspended at about 30-45% in water (303). The cereal resuspension is then crushed with a high shear power processor for about 3 minutes (304). The ground cereal solution is then filtered again (305) and the supernatant is saved. Then, the casein-containing plant cell slurry (disclosed in example 3 and FIG. 1) is added to the supernatant (306) in an amount sufficient to obtain about 0.1-1% of αs1-casein and about 0.5-2% of κ-casein.

The pH of the solution is adjusted to 6.7 (307) by adding suitable acids or bases. Then the casein micelles formation is induced by adding 10% v/v of CaCl₂ solution (0.2 M) and 5% v/v of K₂HPO₄, while the casein solution is kept under strong stirring at room temperature (308). When the addition of chemicals is completed, the solution is kept under strong stirring for about 15 more minutes (309). If necessary, the pH is corrected to 6.7 adding suitable acids or bases. Then, vegetable oils (for instance about 2% canola oil) and saccharides (for instance about 2% sucrose) are added (310), the solution is homogenized for about 5 minutes at room temperature with homogenize (ProScientific) (311). Subsequently, the solution is heated to about 65° C. for about 5 minutes, and then cooled down to about 42° C. (312). When the solution's temperature reaches 42° C., it is maintained at this temperature, and is inoculated with a pre-activated inoculum of lactic bacteria strains (such as Lactobacillus delbrueckii subsp. Bulgaricus and Streptococcus thermophilus) until reach a pH of 4.5±0.1 (2 hours approximately) (313). When the fermentation process is completed, natural flavors and/or food additives are added (314), the product is thoroughly mixed till a creamy texture is formed (315), and the product is then packed in suitable sterile containers (316) and stored in a refrigerator at 4° C. (317).

### Example 9

Yogurt is one of the most widely consumed milk fermented products in the world. Recently, there has been growth in the plant-based yogurt market as these beverages have increasingly become more popular as alternatives to animal-derived yogurt. A key commonality between yogurt derived from animal milk and plant-based yogurt is their fluidic properties, primarily viscosity.

For plant-based yogurt, understanding viscosity is vital during formulation development to meet the expectations of customers. Customers switching from animal-based milks to milk substitutes such as almond beverage, coconut milk, and soy beverage expect similar consistency between the two products. The interactions between small molecules and rheology modifiers in these beverages can cause remarkable changes in their rheological behavior. Understanding and characterizing how the different components alter the rheology of the products can guide and even enhance the development process when formulating new yogurt-like beverages to meet consumer preferences and manufacturing constraints.

Viscosity is a key contributor to the “mouthfeel” characteristic of a beverage. Providing products with viscosity, mouthfeel and taste profiles that meets consumers' needs can lead to enhancing and expanding the markets of plant-based milk beverages and high protein yogurts and fermented drinks.

Having analyzed the physicochemical foundations that determine the texture of a yogurt made from fermented animal milk, the inventors found that caseins play a fundamental role in the texture. Heat treatment, one of the predominant processes of dairy product manufacturing, leads to denaturation of milk proteins and interaction among denatured milk proteins, which may dramatically affect the texture and consistency of yogurt. In this context caseins plays a highly important role, since whey proteins are much more heat-sensitive than casein. The denatured whey proteins interact with each other to form soluble whey proteins that interact with casein micelles to form whey proteins-coated casein micelles. Most whey proteins are denatured during normal heat treatment of yogurt manufacture, during which the formation of disulfide bonds and occurrence of hydrophobic interactions within denatured whey proteins and κ-casein on the surface of casein micelles leads to the formation of whey protein-κ-casein complexes, which largely defines the “mouthfeel” characteristic to a yogurt of an animal origin. The production of caseins (αₛ₁-casein and κ-casein) from a vegetable source (for example carrot cell suspension culture) allows to achieve, through its incorporation into vegetable milks, the formulation of plant-based yogurt with a viscosity and a “mouthfeel” more similar to yogurt of an animal origin. To validate this proposal, different formulations of plant-based yogurt are disclosed in the present application. These plant-based yogurt substitutes comprise cereals (such as oat) with increasing amounts of carrot slurry containing casein. Table 4 lists casein content in the plant-based yogurt substitute of the present invention compared to animal-derived yogurt.

TABLE 4
Total casein content in different products

| | | Total casein |
|---|---|---|
| Denomination | Basic composition | content (% p/v) |
| Sample A | Oat | 0.0% |
| Sample B | Oat + Carrot Slurry | 2.0% |
| Sample C | Yogurt from animal milk | 2.7% |

The texture profile of the samples specified in Table 4. was analyzed using shear stress rheograms as a function of the deformation gradient at a constant temperature of 10° C. Then, the apparent viscosities (cp) of the samples were determined at deformation gradients of: 10, 20, 40, 50, 60, 80, 100, 150 and 200 s⁻¹.

Each sample was thermostatized at 10±0.5° C. for about 30 minutes, using a Rheolab QC (Anton Paar, Germany) concentric cylinder viscometer, with C-LTD80/QC cell and CC27 rotating sensor system. Statgraphics Centurion software was used for statistical analysis. Shear stress diagrams (τ) vs. Deformation Gradient (γ′=DG) varying this in an ascending way from 0 to 200 s⁻¹, and descending from 200 to 0 s⁻¹. From these data, the apparent viscosity (μₐₚ) was calculated as the ratio τ/γ′, at each point (upper curve of the rheogram), and at the requested deformation gradients (by interpolation): 10, 20, 40, 50, 60, 80, 100, 150 and 200 s⁻¹. The test was carried out in duplicates. The average and standard deviation of the calculated apparent viscosities are disclosed. For the statistical analysis, simple ANOVA and a Tukey HSD test were applied to determine differences between treatments (α≤0.05); Statgraphics Centurion software was used. The results for this assay are presented in Table 5.

TABLE 5
Average values, standard deviations and significant differences between samples with
respect to the apparent viscosity (μₐₚ) at 10° C., in centipoise (cP), at the different DGs.

| Viscosity (cP) | Sample A* | Sample B** | Sample C*** | p-value |
|---|---|---|---|---|
| μ10 s⁻¹ | 710.37 ± 9.61 ^{c} | 373.31 ± 12.49 ^{b} | 132.41 ± 5.14 ^{a} | 0.0001 |
| μ20 s⁻¹ | 434.92 ± 12.53 ^{c} | 215.32 ± 0.70 ^{b} | 101.24 ± 5.02 ^{a} | 0.0001 |
| μ40 s⁻¹ | 283.49 ± 0.75 ^{c} | 135.14 ± 1.18 ^{b} | 78.62 ± 3.56 ^{a} | 0.0001 |
| μ50 s⁻¹ | 247.42 ± 3.80 ^{c} | 122.23 ± 1.90 ^{b} | 64.36 ± 2.06 ^{a} | 0.0001 |
| μ60 s⁻¹ | 222.51 ± 1.44 ^{c} | 99.38 ± 0.49 ^{b} | 67.75 ± 3.45 ^{a} | 0.0001 |
| μ80 s⁻¹ | 186.65 ± 0.56 ^{c} | 82.64 ± 0.19 ^{b} | 55.66 ± 0.45 ^{a} | 0.0001 |
| μ100 s⁻¹ | 159.38 ± 0.46 ^{c} | 73.79 ± 0.53 ^{b} | 52.00 ± 0.90 ^{a} | 0.0001 |
| μ150 s⁻¹ | 125.04 ± 0.79 ^{c} | 55.97 ± 0.08 ^{b} | 40.99 ± 0.51 ^{a} | 0.0001 |
| μ200 s⁻¹ | 105.57 ± 0.58 ^{c} | 45.85 ± 0.02 ^{b} | 35.80 ± 0.15 ^{a} | 0.0001 |

*Sample A: Oat
**Sample B: Oat ± Carrot Slurry
***Sample C: Yogurt from animal milk

It is observed that the three samples presented a slightly thixotropic behavior, in the entire range of DG tested, that is, the apparent viscosity decreases with the increase of the same (mechanical treatment on the sample) and with the time of said treatment.

In addition, there are statistically significant differences between the samples, with sample A presenting the highest viscosity in the entire range of DG, which coincides with what was previously stated in the attached table.

The sample with the lowest viscosity, in the entire range of DG, was the sample of skimmed commercial yogurt from animal origin (sample C), with values visibly different from the other samples.

It is important to note that the only difference between sample A and B is the addition of recombinant casein proteins prior to the fermentation process. The results show that the addition of recombinant caseins produced a significant reduction (approximately 60%) in viscosity throughout the DG range, allowing the viscosity of oat-based yogurt to have a viscosity closer to that of yogurt of animal origin.

In 1992, Elejalde and Kokini showed that there is seemingly an excellent power law (linear on log-log scale) correlation between the viscosity at 50 s⁻¹ and thickness perception for a large range of different foods over several orders of magnitude. The apparent viscosity at 50 s⁻¹ was named Kokini Viscosity, and for soft foods, it has been found to relate strongly to the initial texture perception in mouth, such as firmness of yogurts or mayonnaise. Therefore, the inventors focused on to the Kokini viscosity (μ50) in order to properly analyze the mouthfeel of the plant-based yogurt substitute of the present invention. Kokini viscosity represents the behavior of a product during normal chewing. The results indicate that the addition of the αₛ₁-casein κ-casein prior to the fermentation process allowed to reduce the apparent viscosity by more than 100% in the oat-based yogurt. Although there are differences with the apparent viscosity of yogurt of animal origin at μ50 s⁻¹, it is important to note that sample B contains approximately 2% total caseins, while cow's milk contains approximately 2.7%. It is expected that as the dosage of recombinant caseins increases in the formulation of the plant-based yogurt substitute of the present invention, the apparent viscosity and mouthfeel will fully equal or even surpass the values of yogurt of animal origin.

### Example 10

Proteins currently used by the food industry for their emulsifying abilities are mostly derived from milk (or whey), soybean, eggs, etc. These proteins are widely used due to of their commercial availability, high nutritional values, and excellent functional properties. The major drawback of these proteins is that they have all been identified as common food allergens. There are also rising concerns related to dietary restrictions associated with milk and egg proteins, the spread of diseases such as bovine spongiform encephalitis, and multidrug-resistant food-borne pathogens. The ability of a protein to form and stabilize an emulsion droplet is related to its ability to adsorb and unfold rapidly at the nascent oil-water interface (that is, its surface activity).

The most commonly used vegetable emulsifying alternatives in products such as coffee creamers and coffee whiteners are soy lecithin and concentrated soy proteins.

Caseins are good at forming emulsions and giving short-term stability within the homogenizer. Here, the inventors perform a comparative study of the emulsifying capacity of recombinant caseins expressed in carrots, wild type carrot cell slurry, soy lecithin, and concentrated soy proteins.

### Protocol for the Emulsifying Capacity Study:

The emulsifying power of a surfactant is determined by the capacity of an aqueous solution with 2% of surfactant to emulsify the same volume of certain oil. This trial is performed to evaluate proteins, fibers, lecithin or chemical surfactants.
1) In different bickers 50 ml of an aqueous solutions were prepared with 2% of the following emulsifiers:
   Carrot slurry (CS) with αs1 and κ caseins (with different concentrations of total caseins);
   Isolated soy protein (70% purity);
   Isolated pea protein (85% purity);
   Soy lecithin
2) The solutions were homogenized using Ultraturrax® homogenizator for about one minute (intermediate shear force position) or until complete suspension and no presence of particles.
3) Then, 50 ml of sunflower oil were added to each bicker.
4) All the solutions were homogenized using Ultraturrax® homogenizator for about one minute (intermediate shear force position).
5) Each final emulsification was added in two 50 ml falcon tubes. Then the tubes were centrifuged at 3000 rpm for about 10 minutes.
6) After the centrifugation 3 or 4 phases can be found on each tube: emulsified phase (EP) (with or milky aspect), aqueous phase (AP), oil phase (OP) and sedimented phase (SP). The volume of each phase in each tube was measured.
7) The formulation applied for the calculation of the Emulsifying Capacity (EC) is the following:

$$E C = \frac{E P}{Total Volume} \times 100 \\ Total Volume = EP + AP + OP + SP$$

Protocol for Evaluating Emulsion Stability Under High Temperatures

For the applications of vegetal surfactants in formulations like coffee creamers of coffee whiteners it is important to evaluate the stability of the emulsion or surfactant activity at high temperatures. In this study the emulsifications obtained during the trial of emulsifying capacity were incubated at high temperatures to evaluate their stability.

The tubes obtained after the centrifugation in the emulsifying capacity study were incubated in a water bath at 80° C. for about 30 minutes.
1—After the incubation step, the tubes were centrifuged at 3000 rpm for about 10 minutes.
2—The volume of the Emulsified Phases (EP′, under high temp) was measured for each tube.
3—The formulation applied for the calculation of the Emulsifying Stability (ES) is the following:

$$E S = \frac{{EP}^{′}}{E P} \times 100$$

The results of the emulsifying capacity and emulsifying stability of several emulsifiers, including the casein-expressing carrot slurry disclosed in the present application, are described in Table 6.

TABLE 6
Emulsifying capacity (EC) and emulsifying stability
(ES) of casein expressing carrot slurry

| | Tube 1 | | | | Tube 2 | | | | Average | Average |
|---|---|---|---|---|---|---|---|---|---|---|
| Emulsifier | EP | EC | EP’ | ES | EP | EC | EP’ | ES | EC | ES |
| Isolated soy | 23.8 | 48.7 | 22.5 | 94.7 | 23.8 | 47.3 | 25.0 | 105.3 | 48.0 | 100.0 |
| protein | | | | | | | | | | |
| Isolated pea | 21.3 | 47.8 | 17.5 | 82.4 | 21.3 | 47.8 | 20.0 | 94.1 | 47.8 | 88.2 |
| protein | | | | | | | | | | |
| Soy Lecithin | 22.5 | 45.0 | 2.5 | 11.1 | 22.5 | 45.0 | 3.4 | 15.0 | 45.0 | 13.1 |
| CS with Caseins | 27.5 | 55.0 | 30.0 | 109.1 | 27.0 | 54.0 | 29.0 | 107.41 | 54.5 | 108.2 |
| 2% | | | | | | | | | | |

The results of the tests performed show that the transgenic caseins expressed in carrot cells can be applied to fulfill the functions of emulsifiers or food creams. The stability of the emulsions formed at room temperature proved to be 13.5% higher than the one with the best performance (purified soy protein). In addition, it was observed that when faced with heat treatment, the emulsifying capacity of caseins increased, unlike other emulsifiers such as soy lecithin. following heat stress, the transgenic casein emulsions showed stability which is 8.2% higher than purified soy protein.

### Example 11

The development of plant-based fermented dairy substitutes, such as cheese and yogurt, has made great progress in recent years. By extracting proteins from vegetable sources such as peas, oats, almonds, among others, and with the addition of fibers and vegetable texturizers, it is possible to reproduce textures similar to the different types of cheeses of animal origin. However, it has not yet been possible to develop plant sources that provide common aromas and flavors of dairy products of animal origin.

The flavor of a fermented dairy food of animal origin, in combination with the overall appearance and its texture, is crucial for consumer's selection and preference. Consequently, the importance of studying dairy flavors is principally related to both the acceptance within the marketplace and the perception of a dairy flavor by the consumer. It is known that the flavor preference of the consumer is motivated by the stimulation of human chemical senses, particularly those sensing odor (aroma) and taste. Specifically, the perception of flavor is mainly driven by the combination of active volatile compounds perceived in the ortho-nasal and/or retro-nasal cavity. A precise chemical characterization of the mixture of potential stimulants in food plays an important role in investigating olfactory perception and to evaluate food flavor.

The biochemical processes which lead to the synthesis of volatile compounds in a fermented dairy produced by the fermentation of cow milk are extremely complex. It is known that the volatile compounds identified in cheese are mainly the products of lipolysis, proteolysis, metabolism of residual lactose, lactate, and citrate. They also include metabolism of free fatty acids (FFAs), and free amino acids (FAAs).

In a very detailed study conducted in 2001 by Mireille Yvon and Liesbeth Rijnen, it was described that the catabolism of FAAs produces mainly aldehydes, alcohols, carboxylic acids, amines, and sulfur compounds, and the unique source of FAAs are caseins. Aromatic amino acids, branched-chain amino acids, methionine, and aspartic acid are converted into α-keto acids by a transamination reaction, catalyzed by amino acid aminotransferase. The resulting α-keto acids are then further degraded to branched-chain and aromatic aldehydes, acyl-CoA, hydroxy acids, and methanethiol. The transamination of valine, isoleucine, and leucine leads to the production of 2-methylpropanal, 2-methylbutanal, and 3-methylbutanal, respectively. Aspartic acid can be converted by transamination into oxaloacetate and further into acetoin, diacetyl, or 2,3-butanediol. Aldehydes can be converted to their corresponding alcohols by alcohol dehydrogenase, or oxidized to their corresponding carboxylic acids by aldehyde dehydrogenase. The metabolism of FAAs by decarboxylation can produce amines, which are not associated with good quality cheese, due to their potentially adverse health effects and often poor flavor. In addition, catabolism of FAAs can be initiated by elimination reactions, catalyzed by amino acid lyase, which cleave the side chain of amino acids. This pathway leads to the synthesis of phenol and indole from the metabolism of aromatic amino acids, and to the production of methanethiol from methionine.

In this way, obtaining casein peptides from a vegetable source is an opportunity to generate the spectrum of volatile organics mentioned above and incorporate them into plant-based dairy substitutes for improving the organoleptic properties of those products, mainly flavor and aroma.

To validate this notion, the inventors carried out several tests to determine volatile organic compounds by Gas Chromatography associated with Mass Spectrometry (GC-MS) in different samples of coagulated and fermented recombinant caseins with lactic bacteria.

### Samples Preparation:

1. First, the pre-activation of lactic bacteria was conducted. 0.1 g of inoculum of Hansen's lactic bacteria TCC-4® (Chr. Hansen—composition: Lactobacillus delbrueckii subsp. Bulgaricus and Streptococcus thermophilus) and 1 g of sucrose were dissolved in 20 ml of water. The solution was kept in a water bath at about 34° C. for about 3 hours.
2. Then, 3 g of protein (pea protein or carrot slurry containing α_{S1}-casein and κ-casein, depending on the sample) were weighed and 10 ml of water and 5 ml of pre-activated ferment were added.
3. The protein and lactic bacteria mixture were placed in a Petri dish and tape-sealed at the edges.
4. Finally, the mixture was placed in an oven at about 34° C. for 24 hours.

After the fermentation process, 1.6 g of each sample was placed in a 3.0 mL vial suitable for HEAD SPACE analysis. Subsequently, the volatiles present in the “headspace” of the solutions were determined by Solid Phase Microextraction (SPME). For this, the compounds in equilibrium were adsorbed on a Carboxen/Polydimethylsiloxane (CAR/PDMS) fiber (75 μm-SUPELCO) with manual holder, for a period of 30 minutes at room temperature (5° C.±1° C.). Then, the compounds were desorbed in the injector of the chromatograph at a temperature of 250° C. for about 2 minutes.

Chromatographic analysis was carried out on a Thermo-TRACE 1300 chromatograph equipped with an HP-5 ms column (0.25 μm, 0.25 mm, 60 m). The temperature program and the flow used are detailed below in Table 7:

TABLE 7
Program for chromatographic analysis

| HEAT RATE | TEMPERATURE | TIME |
|---|---|---|
| (° C./min) | (° C.) | (minutes) |
| | 50 | 5 |
| 15 | 250 | 15 |

| | |
|---|---|
| Flow Rate: 2 ml/min | Injection: Split; split ratio = 10 |

The detection of the compounds at the exit of the chromatograph was performed with a Thermo-ISQ-LT mass spectrometer. The temperature of the transfer line was 270° C. and ionization by electron impact (70 Ev; 275° C.) in full scan mode (35-500 m/z; 0.2 sec).

The identification of the peaks was carried out by comparison with the spectra of the Libraries of the NIST MS Search 2.0 program.

The compounds were listed in order of the retention time (R.T., in minutes), and are designated as having a Zero peak area (0), or a small (S), medium (M), or large (L) average peak area. The compounds and retention time are presented in Table 8.

TABLE 8
Organic compounds detected in samples of coagulated and
fermented pea proteins and recombinant caseins

| Appex | | Relative Quantity | |
|---|---|---|---|
| R.T. | Organic Compound | Pea Proteins | Caseins |
| 3,1 | 1-Propen-2-ol, acetate | S | 0 |
| 3,68 | Acetic acid | 0 | L |
| 3,7 | 2,3-Butanedione | L | 0 |
| 4,56 | Butanal, 3-methyl- | 0 | M |
| 4,74 | Formic acid, 2-propenyl ester | 0 | S |
| 5,39 | 2,3-Pentanedione | L | 0 |
| 5,76 | 2-Butanone, 3-hydroxy- | L | M |
| 6,54 | 1-Butanol, 3-methyl- | 0 | L |
| 6,96 | Disulfide, dimethyl | 0 | S |
| 7,82 | 1-Pentanol | M | 0 |
| 8,29 | 2,3-Butanediol | 0 | S |
| 8,64 | Butanoic acid | 0 | L |
| 8,82 | Hexanal | M | 0 |
| 10,06 | Butanoic acid, 3-methyl- | M | L |
| 10,29 | Butanoic acid, 2-methyl- | 0 | M |
| 10,64 | 1-Hexanol | L | 0 |
| 10,82 | 1-Butanol, 3-methyl-, acetate | 0 | S |
| 11,11 | 2-Heptanone | L | M |
| 12,17 | 1,11-Undecanedicarboxylic acid | 0 | S |
| 12,25 | 4-Heptanol, 2,6-dimethyl- | 0 | L |
| 12,41 | 2-Heptenal | M | 0 |
| 12,53 | Benzaldehyde | M | 0 |
| 12,62 | Ethyl glycolate | M | 0 |
| 12,78 | 1-Octen-3-ol | L | 0 |
| 12,99 | Furan, 2-pentyl- | L | 0 |
| 13,25 | 2-Ethyl-2-hexenal | S | 0 |
| 13,64 | 17-Octadecynoic acid | S | 0 |
| 13,97 | 2-Octenal | S | 0 |
| 14,38 | 2-Nonanone | S | 0 |
| 14,55 | Nonanal | M | 0 |
| 15,29 | Octanoic Acid | 0 | L |
| 15,32 | 1-Nonanol | S | 0 |
| 15,57 | 2-Decanone | M | 0 |
| 16,38 | 1-Decanol | 0 | M |
| | 2,6,6-Trimethylcyclohexa-1,4- | | |
| 16,98 | dienecarbaldehyde | S | 0 |
| 17,06 | 1,3-Diacetin | S | 0 |
| 17,23 | n-Decanoic acid | 0 | M |
| 18,25 | Formic acid, dodecyl ester | 0 | S |
| 20,27 | cis-11-Eicosenoic acid | S | 0 |

The results of the organic compounds' spectra of both samples indicate that the volatility profile obtained by fermenting vegetable proteins (such as pea proteins) and caseins are completely different. These two types of fermented proteins substantially differ in aromas and flavors.

It is important to highlight that most of the volatile organic components found in the spectrum of coagulated and fermented recombinant caseins can be found in the scientific literature of studies of volatile organics in dairy products of animal origin.

In 2001, Valero, Sanz, and Martinez-Castro conducted a study for the determination of volatile compounds by GC in different commercial ripened cheeses of animal origin, such as La Serena, Camembert and Cabrales. This work reports the presence and relevance of several volatile organic compounds that were also detected in the assay disclosed herein on fermented recombinant caseins. Among those volatile organic compounds are Acetic acid, 3-methyl-Butanal, Butanoic acid, 2-methyl-Butanoic acid and Decanoic acid.

In 2015, Fuchsmann, Stern, Brugger, and Breme conducted an analysis of GC using headspace solid-phase microextraction 4 gas chromatography-mass spectrometry/pulsed flame-photometric detection, and gas 5 chromatography-olfactometry to identify and quantitate volatile compound in three differently fabricated commercial Swiss 3 Tilsit cheeses. The researchers found that buttery-cheesy odor notes were attributed to certain molecules like butanoic acid, an organic volatile compound found in relatively large quantity in the fermented recombinant caseins sample. The group also discuss the relevance and contribution of other organic compounds to the cheesy flavor and aroma. Such compounds, like 2-methyl-Butanoic acid and Octanoic Acid are also found in the GC trial disclosed herein.

In 1993, Bosset and Gauch, performed a detailed study of volatile organic compounds by GC in six ripe cheeses labelled with an “appellation d'origine controlee”: Parmigiano Reggiano (ripened for 28 months) and Fontina (3.5 months) from Italy; Mahón (3 months) from Spain; Comté (6.5 months) and Beaufort (7 months) from France; and Appenzeller (3.5 months) from Switzerland. The results published in this article indicated the presence of two volatile compounds also found in the samples of fermented recombinant caseins: 3-methyl-1-butanol and 3-methyl-acetate-1-butanol.

Based on the results obtained in the analysis of volatile organic compounds of recombinant caseins expressed in carrot cells following fermentation with lactic bacteria, the inventors conclude that the process of obtaining caseins from a vegetable source represents an innovative tool that allows adding notes of aromas and flavors from dairy products of animal origin to plant-based formulations.

1.-42. (canceled)

43. A plant-based dairy substitute comprising:
a. a slurry of transgenic plant cells expressing at least one form of casein;
b. water;
c. at least one chemical;
d. at least one food additive;
e. at least one vegetable oil;
f. at least one saccharide;
g. at least one vegetable protein; and
h. at least one strain of lactic bacteria;
wherein said slurry is configured to be fermented by said lactic bacteria, thereby producing a plant-based dairy substitute characterized by organoleptic and physicochemical properties characteristic of dairy products of animal origin, wherein said slurry is characterized as comprising disrupted plant cells.

44. The plant-based dairy substitute of claim 43, characterized by at least one of the following:
a. said substitute is selected from a group consisting of cheese, yogurt, cream, custard, ice cream, coffee creamers and emulsifiers for the food industry;
b. said at least one form of casein is selected from a group consisting of bovine alpha S1 casein, bovine kappa casein, bovine alpha S2 casein, bovine beta-casein, and any combination thereof;
c. said transgenic plant cells are selected from a group consisting of cell suspension cultures, hairy root cultures, transgenic plants and any combination thereof;
d. said transgenic plant cells are selected from a group consisting of carrot cells, rice cells, beetroot cells, tobacco cells, potato cells, sweet potato cells, tomato cells, Arabidopsis cells, Nicotiana benthamiana cells, cassava cells, kohlrabi cells, parsley cells, horseradish cells, jackfruit cells, Anchusa officinalis cells and any combination thereof;
e. said at least one chemical is selected from the group consisting of calcium salts, potassium phosphate dibasic, monobasic potassium phosphate and any combination thereof;
f. said at least one food additive is selected from the group consisting of stabilizers, emulsifiers, anticaking agents, salts, yeast extract, flavorings, antifoaming agents, antioxidants, bulking agents, colorants, humectants, preservatives, sweeteners, vitamins, hydrocolloids, thickeners and any combination thereof;
g. said at least one vegetable oil is selected from a group consisting of coconut oil, canola oil, corn oil, olive oil, cottonseed oil, palm oil, peanut oil, sesame oil, soybean oil, grapeseed oil sunflower oil and any combination thereof;
h. said vegetable oil comprises about 4%-15% of said plant-based dairy substitute;
i. said at least one vegetable protein is selected from a group consisting of nuts, grains, seeds, fruits, tubers and any combination thereof;
j. said organoleptic properties are selected from a group consisting of texture, consistency, appearance, taste, odor, flavor, aroma, touch, mouthfeel and any combination thereof;
k. said physicochemical properties are selected from a group consisting of strength, firmness, tightness, resilience, rheological parameters, moisture content, viscosity, hardness, springiness, adhesiveness, cohesiveness, elasticity, chewability, degradation rate, solvation, porosity, electrical charge and any combination thereof;
l. said plant-based dairy substitute comprises at least 5 milligrams beta-carotene per 1 Liter;
m. said transgenic plant cells are configured to be spray-dried into a plant cell powder, prior to forming said slurry;
n. said at least one vegetable protein comprises about 1%-10% of said plant-based substitute;
o. at least one saccharide is selected from a group consisting of starch, modified starch, non-modified starch, corn starch, potato starch, rice starch, tapioca starch, maltodextrin, inulin, glucose, sucrose, fructose, dextrose, inverted sugar and any combination thereof;
p. said at least one strain of lactic bacteria is selected from a group consisting of Aerococcus, Enterococcus, Lactobacillus, Lactococcus, Leuconostoc, Oenococcus, Pediococcus, Streptococcus and any combination thereof.

45. The plant-based dairy substitute of claim 44, wherein said at least one vegetable protein is selected from a group consisting of cashew, almonds, peanuts, walnuts, brazil nuts, rice, wheat, oat, rye, corn, quinoa, lentil, sesame, chia, pea, chickpea, lupine, soybean, fava bean, mung bean, pumpkin seeds, sunflower seeds, flaxseeds, potato, cassava, yam and any combination thereof.

46. The plant-based dairy substitute of claim 44, wherein said lactic bacteria are selected from the group consisting of Streptococcus thermophilus, Lactobacillus helveticus, Lactobacillus bulgaricus, Lactococcus lactis, Lactococcus cremoris, Lactococcus diacetylactis, Lactobacillus acidophilus, Lactobacillus casei, Lactobacillus lactis, Lactobacillus helveticus, Lactobacillus bulgaricus Bifidobacterium and any combination thereof.

47. The plant-based dairy substitute of claim 44, wherein said plant cell powder is storable without refrigeration at about 25° C. for about 6 months.

48. A method for producing a plant-based cheese substitute comprising steps of:
a. obtaining a slurry of transgenic plant cells expressing at least one form of casein;
b. dissolving said slurry of transgenic plant cells;
c. forming casein micelle solution;
d. stirring said casein micelle solution;
e. incubating said casein micelle solution with at least one rennet-forming enzymes to form a rennet;
f. filtering said rennet;
g. resuspending said rennet in water to form a solution;
h. adding at least one food ingredient to said solution;
i. mixing said solution;
j. adding at least one vegetable oil to said solution;
k. incubating said solution with at least one strain of pre-activated lactic bacteria to form a fermented preparation;
l. sieving said fermented preparation;
m. adding at least one food additive to said fermented preparation;
n. homogenizing said fermented preparation; and
o. packing said fermented preparation;
wherein, said plant-based cheese substitute is characterized by organoleptic properties and physicochemical properties characteristic of conventional cheese products of animal origin, wherein said slurry is characterized as comprising disrupted plant cells.

49. The method of claim 48, characterized by at least one of the following:
a. said at least one form of casein is selected from a group consisting of bovine alpha S1 casein, bovine kappa casein, bovine alpha S2 casein, bovine beta-casein, and any combination thereof;
b. said plant cells are selected from a group consisting of carrot cells, rice cells, beetroot cells, tobacco cells, potato cells, sweet potato cells, tomato cells, Arabidopsis cells, Nicotiana benthamiana cells, cassava cells, kohlrabi cells, parsley cells, horseradish cells, jackfruit cells, Anchusa officinalis cells and any combination thereof;
c. said forming casein micelle solution is obtained by the addition of about 10% 0.2 M CaCl₂ and about 5% 0.2M K₂HPO₄;
d. said at least one rennet-forming enzymes is chymosin;
e. said at least one food ingredient is selected from a group consisting of plant fibers, starch, saccharides, yeast extracts, amino acids, proteins from a plant origin and any combination thereof;
f. said at least one vegetable oil is selected from a group consisting of coconut oil, canola oil, corn oil, olive oil, cottonseed oil, palm oil, peanut oil, sesame oil, soybean oil, grapeseed oil sunflower oil and any combination thereof;
g. said at least one strain of pre-activated lactic bacteria are selected from the group consisting of Streptococcus thermophilus, Lactobacillus helveticus, Lactobacillus bulgaricus, Lactococcus lactis, Lactococcus cremoris, Lactococcus diacetylactis, Lactobacillus acidophilus, Lactobacillus casei, Lactobacillus lactis, Lactobacillus helveticus, Lactobacillus bulgaricus Bifidobacterium and any combination thereof;
h. said organoleptic properties are selected from a group consisting of texture, consistency, appearance, taste, odor, flavor, aroma, touch, mouthfeel and any combination thereof;
i. said physicochemical properties are selected from a group consisting of strength, firmness, tightness, resilience, rheological parameters, moisture content, viscosity, hardness, springiness, adhesiveness, cohesiveness, elasticity, chewability, degradation rate, solvation, porosity, electrical charge and any combination thereof.

50. A method for producing a plant-based yogurt substitute comprising steps of:
a. obtaining a slurry of transgenic plant cells expressing at least one form of casein;
b. adding said slurry to a supernatant of pre-hydrated, pre-filtered cereal suspension;
c. forming casein micelle solution;
d. stirring said casein micelle solution;
e. adding at least one vegetable oil and at least one saccharide;
f. homogenizing said solution;
g. heating said solution to about 65° C.;
h. cooling down said solution to about 42° C.;
i. incubating said solution with at least one strain of pre-activated lactic bacteria to form a fermented preparation;
j. adding at least one food additive to said fermented preparation;
k. mixing said fermented preparation; and
l. packing said fermented preparation,
wherein, said plant-based yogurt substitute is characterized by organoleptic properties and physicochemical properties characteristic of conventional yogurt products of animal origin, wherein said slurry is characterized as comprising disrupted plant cells.

51. The method of claim 50, characterized by at least one of the following:
a. said at least one form of casein is selected from a group consisting of bovine alpha S1 casein, bovine kappa casein, bovine alpha S2 casein, bovine beta-casein, and any combination thereof;
b. said plant cells are selected from a group consisting of carrot cells, rice cells, beetroot cells, tobacco cells, potato cells, sweet potato cells, tomato cells, Arabidopsis cells, Nicotiana benthamiana cells, cassava cells, kohlrabi cells, parsley cells, horseradish cells, jackfruit cells, Anchusa officinalis cells and any combination thereof;
c. said cereal is selected from a group consisting of oat, wheat rye, spelt, triticale and any combination thereof;
d. said forming casein micelle solution is obtained by the addition of about 10% 0.2 M CaCl₂ and about 5% 0.2M K₂HPO₄;
e. said at least one vegetable oil is selected from a group consisting of coconut oil, canola oil, corn oil, olive oil, cottonseed oil, palm oil, peanut oil, sesame oil, soybean oil, grapeseed oil sunflower oil and any combination thereof;
f. said at least one saccharide is selected from a group consisting of starch, modified starch, non-modified starch, corn starch, potato starch, rice starch, tapioca starch, maltodextrin, inulin, glucose, sucrose, fructose, dextrose, inverted sugar and any combination thereof;
g. said at least one strain of pre-activated lactic bacteria are selected from the group consisting of Streptococcus thermophilus, Lactobacillus helveticus, Lactobacillus bulgaricus, Lactococcus lactis, Lactococcus cremoris, Lactococcus diacetylactis, Lactobacillus acidophilus, Lactobacillus casei, Lactobacillus lactis, Lactobacillus helveticus, Lactobacillus bulgaricus Bifidobacterium and any combination thereof;
h. said at least one food additive is a flavoring;
i. said organoleptic properties are selected from a group consisting of texture, consistency, appearance, taste, odor, flavor, aroma, touch, mouthfeel and any combination thereof.
j. said physicochemical properties are selected from a group consisting of strength, firmness, tightness, resilience, rheological parameters, moisture content, viscosity, hardness, springiness, adhesiveness, cohesiveness, elasticity, chewability, degradation rate, solvation, porosity, electrical charge and any combination thereof.

52. A slurry comprising disrupted transgenic plant cells expressing at least one form of casein for use in the production of foodstuffs, food ingredients and beverages.


SEQUENCE LISTING (4 sequences)

SEQ ID NO: 1 - DNA, 645 bp, Bos taurus
  misc_feature (1)..(645)
atgaaacttctcatccttacctgtcttgtggctgttgctcttgctaggcctaaacatcctatcaagcaccaaggactccctcaagaagtcctcaatgaaaatttactcaggttttttgtggcaccttttccagaagtgtttggaaaggagaaggtcaatgaactgagcaaggatattgggagtgaatcaactgaggatcaagccatggaagatattaagcaaatggaagctgaaagcatttcgtcaagtgaggaaattgttcccaatagtgttgagcagaagcacattcaaaaggaagatgtgccctctgagcgttacctgggttatctggaacagcttctcagactgaaaaaatacaaagtaccccagctggaaattgttcccaatagtgctgaggaacgacttcacagtatgaaagagggaatccatgcccaacagaaagaacctatgataggagtgaatcaggaactggcctacttctaccctgagcttttcagacaattctaccagctggatgcctatccatctggtgcctggtattacgttccactaggcacacaatacactgatgccccatcattctctgacatccctaatcctattggctctgagaacagtgaaaagactactatgccactgtggtga

SEQ ID NO: 2 - DNA, 573 bp, Bos taurus
  misc_feature (1)..(573)
atgatgaagagttttttcctagttgtgactatcctggcattaaccctgccatttttgggtgcccaggagcaaaaccaagaacaaccaatacgctgtgagaaagatgaaagattcttcagtgacaaaatagccaaatatatcccaattcagtatgtgctgagtaggtatcctagttatggactcaattactaccaacagaaaccagttgcactaattaataatcaatttctgccatacccatattatgcaaagccagctgcagttaggtcacctgcccaaattcttcaatggcaagttttgtcaaatactgtgcctgccaagtcctgccaagcccagccaactaccatggcacgtcacccacacccacatttatcatttatggccattccaccaaagaaaaatcaggataaaacagaaatccctaccatcaataccattgctagtggtgagcctacaagtacacctaccatcgaagcagtagagagcactgtagctactctagaagcttctccagaagttattgagagcccttgagagcccaacacagtccaagttacttcaactgcggtctaa

SEQ ID NO: 3 - DNA, 645 bp, Bos taurus
  misc_feature (1)..(645)
atgaagttgctcatcctgacttgccttgtggctgttgctcttgctaggccaaagcaccctattaagcaccagggacttccacaagaggtgctcaacgagaacctcctcagattcttcgtggccccatttccagaggtgttcggcaaagagaaggtcaacgagctgtctaaggatatcggctctgagtctactgaggaccaggccatggaagatatcaagcagatggaagccgagagcatcagcagctctgaggaaattgtgccaaacagcgttgagcagaagcacatccagaaagaggatgtcccaagcgagagataccttggatacctcgagcagcttctccgcctcaagaagtataaggttccacagctcgagatcgtgcccaactctgctgaagaaaggctccactctatgaaggaaggtatccacgctcagcagaaagaacccatgatcggagtgaatcaagagctggcctacttctacccagagctgttcaggcagttctaccagcttgatgcttacccatctggcgcttggtactacgttccacttggaactcagtacaccgacgctccaagcttcagcgatatcccaaatccaatcgggtccgagaacagcgaaaagactactatgccactctggtaa

SEQ ID NO: 4 - DNA, 573 bp, Bos taurus
  misc_feature (1)..(573)
atgatgaagtcgttctttctcgtggtgaccatcctcgctcttactcttccattccttggtgcccaagagcagaatcaagagcagccaattaggtgcgagaaggacgagagattcttcagcgataagatcgccaagtacatccccatccagtacgtgctgtctcgctatccatcttacggcctcaattactaccagcagaagccagtcgctctcatcaacaaccagttcttgccctatccgtactacgctaagccagctgctgttagatctccagctcagattctccagtggcaggttctctctaataccgtgccagctaagtcttgccaggctcagccaactactatggctagacatccacatccgcacctgagcttcatggctattccaccaaagaagaaccaggacaagaccgagattcccactatcaacaccattgctagcggagagccaacttctaccccaaccattgaggctgttgagtctactgtggctactctcgaggcttctccagaggttatcgagtctccaccagagatcaacactgtgcaggttacctctaccgccgtgtaa